(19) United States
(12) Patent Application Publication
(10) Pub. No.: US 2023/0383341 A1
(43) Pub. Date: Nov. 30, 2023

(54) GENE PROMOTERS FOR USE IN PROKARYOTIC CELLS

(71) Applicants: ExxonMobil Technology and Engineering Company, Annandale, NJ (US); University of Georgia Research Foundation, Inc., Athens, GA (US)

(72) Inventors: Yuchen Liu, Annandale, NJ (US); William B. Whitman, Athens, GA (US); Taiwo S. Akinyemi, Athens, GA (US); Zhe Lyu, Pearland, TX (US); Nana Shao, Athens, GA (US)

(21) Appl. No.: 17/752,338

(22) Filed: May 24, 2022

(51) Int. Cl. C12Q 1/6865; C12N 15/74

(52) U.S. Cl. CPC C12Q 1/6865; C12N 15/74; C12N 2510/02

(57) ABSTRACT

The present invention provides an isolated DNA molecule suitable for use as, or in, a phosphate responsive inducible promoter. The invention also provides expression cassettes comprising the promoter operably linked to a gene. The invention further provides vectors and prokaryotic host cells, for expressing a protein encoded by the gene; methods for transforming prokaryotic cells; and methods of inducing expression of an exogenous gene in a prokaryotic cell culture, the method including lowering the levels of inorganic phosphates in the culture.

### CROSS-REFERENCE TO RELATED APPLICATION

This application claims the priority benefit of U.S. Ser. No. 63/202,230, filed Jun. 2, 2021, which is hereby incorporated by reference in its entirety.

### REFERENCE TO A SEQUENCE LISTING

This application contains references to nucleic acid sequences which have been submitted concurrently herewith as a sequence listing file. The aforementioned sequence listing is hereby incorporated by reference in its entirety.

### FIELD OF THE INVENTION

The present invention relates to gene regulatory elements, particularly promoters, for use in gene expression in prokaryotes, particularly methanogenic archaea.

### BACKGROUND OF THE INVENTION

Methanogenic archaea (also known as methanogens) are a large and diverse group of strict anaerobes capable of a specialized metabolism that produces large quantities of methane. They employ unusual enzymes and coenzymes to metabolize a limited number of simple substrates such as CO₂, formate, methylated C₁ compounds and acetate to meet their energy and carbon needs. These coenzymes function either as C₁ or electron carriers and can perform reactions with extremely low redox potentials. Methanogens have large amounts of intracellular Fe, and their genomes encode large numbers of 4Fe-4S motifs. Metabolic models exist that examine the relationships between CO₂ and H₂ consumption rates, CH₄ production rates as well as carbon flux to biomass.

### SUMMARY OF THE INVENTION

According to a first aspect, provided herein is an isolated DNA molecule, comprising a sequence having at least about 80% identity to at least about 80 contiguous nucleotides of SEQ ID No. 1.

According to a second aspect, there is also provided an isolated DNA molecule comprising a sequence having at least about 80% identity to at least about 200 contiguous nucleotides of SEQ ID No. 2.

According to a third aspect, there is also provided an expression cassette comprising: a promoter comprising the DNA molecule according to the first aspect or the second aspect, and a gene encoding a polypeptide or a functional RNA sequence, wherein the gene is operably linked to the promoter.

According to a fourth aspect, there is provided a vector comprising the expression cassette according to the third aspect.

According to a fifth aspect, there is provided a prokaryotic host cell comprising the expression cassette according to the third aspect.

According to a sixth aspect, there is provided a cell culture comprising prokaryotic host cells according to the fifth aspect.

According to a seventh aspect, there is provided a method for inducing expression of an exogenous gene in a prokaryotic cell culture according to the sixth aspect, the method comprising lowering the concentration of inorganic phosphates in the culture from an initial concentration.

According to an eighth aspect, there is provided a method for transforming a prokaryotic cell, the method comprising: introducing the vector according to the fourth aspect into the prokaryotic cell; and selecting for a transformed prokaryotic cell.

According to a ninth aspect, there is provided a method for co-transforming a prokaryotic cell, the method comprising: introducing the expression cassette according to the third aspect and a nucleic acid sequence encoding a selectable marker into the prokaryotic cell; and selecting for the presence of the selectable marker in a transformed prokaryotic cell to provide a prokaryotic cell transformed with the expression cassette.

These and other features and attributes of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings, wherein:

FIG. 1 shows the nucleotide sequence of the 243 bp intergenic sequence between MMP1094 and MMP1095 containing the pst promoter. The sequence contains two putative factor B recognition element (BRE) and TATA boxes, named cis-1 and cis-2 respectively. cis-2 is 23 bp from the transcription start site (TSS) while cis-1 is 131 bp upstream. The TSS was determined by dRNA-seq to be 22 bp from the start codon³⁷; hence yielding a 22 bp 5′ untranslated region (UTR). The 25 bp AT-rich region is conserved in related methanococci and begins at −93 bp. The spacer length between the putative RBS and the start codon is 6 bp. Direct repeats were present in the AT-rich region, and an inverted repeat was present immediately downstream the TATA box. See the Methods section of the Examples for how the Ppst was cloned into an mCherry reporter plasmid.

FIG. 2 shows multiple sequence alignment (MSA) of intergenic DNA sequences upstream of the pst operon in the genus Methanococcus. MSA was done using the Clustal omega program. The resulting aligned sequence was graphically enhanced by the ESPript program (http://espript.ibcp.fr/ESPript/ESPript/).

FIG. 3 shows a map of the pMEV5 shuttle vector with Ppst and the gene encoding mCherry. The mCherry gene was cloned in-frame for expression under the control of Ppst. The plasmid contains ColE1 ori, an origin of replication in E. coli; ORFLESS1, a possible origin of replication in Methanococcus; RBS, a methanococcal ribosome binding site; Tsl terminator, a synthetic transcription terminator; Pmcr, the promoter of the methylcoenzyme M reductase gene from Methanococcus voltae; pur, a puromycin resistance cassette for positive selection in M. maripaludis; Tmcr, the terminator of the methylcoenzyme M reductase gene from M. voltae; amp, the ampicillin resistance cassette for positive selection in E. coli.

FIGS. 4A-4D show changes in mCherry expression and phosphate concentration during growth in limiting and replete phosphate media. Growth on formate media for FIG. 4A 243-bp Ppst, FIG. 4B 93-bp Ppst, and FIG. 4C the constitutive promoter PhmvA. FIG. 4D growth of the 93-bp Ppst construct in H₂/CO₂ media. The mCherry expression level or NFU, culture optical density at 600 nm or OD₆₀₀ and phosphate concentration at low, initial 40 μM, phosphate (∘, open circles), and at high, 800 μM phosphate concentration (▴, closed triangles). NFU or normalized fluorescence units is the empirical mCherry fluorescence divided by the OD₆₀₀. The phosphate concentrations at high phosphate were all greater than 800 μM and are not shown. Error bars represent standard deviation of three replicates. Error bars smaller than the symbols are not shown.

FIG. 5 shows the role of the AT-rich region on phosphate-dependent expression. mCherry expression from truncations of the Ppst that remove parts of the AT-rich region. Top panel: expression was determined following growth at early stationary phase on limiting (80 μM) and high (800 μM) phosphate. NFU is the empirical mCherry fluorescence divided by the OD₆₀₀. Error bars represent standard deviation of three replicates. Bottom panel: fold change is the ratio of the NFU in limiting to NFU in high phosphate concentrations.

FIGS. 6A-6C show regulation of mCherry expression with 93 bp Ppst in response to Pᵢ levels. The host cells were cultivated in the formate medium at the indicated Pi concentrations. FIG. 6A the cell growth was monitored by measuring optical density at 600 nm (OD₆₀₀). FIG. 6B the mCherry expression levels were measured by quantitative real-time PCR (qRT-PCR) and depicted by mCherry mRNA copy numbers per 0.5 ng of purified total RNA. FIG. 6C the mCherry protein levels were represented by NFU. The early exponential (EEX), late exponential (LEX), early stationary (EST), and late stationary (LST) growth phases represent time points at 14, 20, 22-26, and 36 h, respectively. Error bars represent standard deviation of three replicates. Error bars smaller than symbols are not shown.

FIG. 7 shows the role of the inoculum growth conditions in the regulation of mCherry expression with 93 bp Ppst. The host cells were cultivated in the formate medium at indicated Pi concentrations with inoculum grown in 800 μM (HPi) or 80 μM (LPi) phosphate. The mCherry protein levels were represented by NFU. The early exponential (EEX), late exponential (LEX), early stationary (EST), and late stationary (LST) growth phases represent time points at 14, 20, 22-26, and 36 h, respectively. Error bars represent standard deviation of three replicates.

FIGS. 8A-8B show the effect of puromycin on cell growth and mCherry expression. FIG. 8A growth of the M. maripaludis 93 bp Ppst construct with different concentrations of puromycin dihydrochloride (Sigma, #P8833), 0 μg/ml (●), 1.25 μg/ml (♦), 2.5 μg/ml (▴). The growth media contained low (80 μM, broken lines) or high (800 μM, solid lines) phosphate concentrations. FIG. 8B the mCherry protein levels was determined at both low (80 PM) and high (800 μM) phosphate concentrations in early stationary phase cultures. Error bars represent standard deviation of three replicates. Error bars smaller than symbols are not shown.

FIGS. 9A-9B show the effect of different BREs on mCherry expression. FIG. 9A mutations in the BRE tested in the 93 bp Ppst construct. BRE-pst is the native BRE of the 93 bp Ppst. BRE-slp contains the BRE from the highly expressed gene slp. BRE-1338 and BRE-0466 contain the BREs from the weakly expressed genes MMP1338 and MMP0466, respectively. NFU fold changes were calculated as the NFU following growth with low phosphate (LPi=80 μM) divided by the NFU following growth with high phosphate (HPi=800 μM). FIG. 9B the mCherry protein levels from the normalized fluorescence units (NFU) was determined at both low (80 μM) and high (800 μM) phosphate concentrations in early stationary phase cultures. Error bars represent standard deviation of three replicates.

FIG. 10 shows the secondary mRNA structures of Ppst 5′ UTR (UTR-pst), two mutated variants of UTR-pst (UTR-pst-5S and UTR-pst-4S) and four 5′ UTR from differently expressed genes (slp, hisA, mcrB and MMP1338) predicted by the RNAfold program (http://ma.tbi.univie.ac.at//cgi-bin/RNAWebSuite/RNAfold.cgi). The start of transcription is labeled the 5′ end while the last nucleotide of the start codon is labeled the 3′ end. The start codon AUG is boxed. The predicted RBS similar to the highly conserved 5′-GGTGG-3′ sequence⁴⁸ is also boxed.

FIGS. 11A-11B show regulation of mCherry expressions with different 5′ UTR sequences. FIG. 11A mutations of the UTR tested in 93 bp Ppst. UTR-pst has the native sequence. UTR-slp, UTR-his and UTR-mcr comprise the 5′ UTRs from the strongly expressed genes slp, hmmA and mcrB, respectively. UTR-1338 has the 5′ UTR from the weakly expressed gene MMP1338. UTR-pst-4S and UTR-pst-5S are variants of UTR-pst with spacer length of 4 bp and 5 bp, respectively. NFU fold changes are calculated by the NFU under LPi condition divided by the NFU under HPi condition. FIG. 11B the mCherry protein levels were determined at both low (80 μM) and high (800 μM) phosphate concentrations in early stationary phase cultures. Error bars represent standard deviation of three replicates.

FIGS. 12A-12B show the effect of mutations of the start codon on mCherry expression. FIG. 12A mutations of the start codon tested in 93 bp Ppst. NFU fold changes are calculated by the NFU under LPi condition divided by the NFU under HPi condition. FIG. 12B mCherry protein levels were determined at both low (80 μM) and high (800 μM) phosphate concentrations in early stationary phase cultures. Error bars represent standard deviation of three replicates.

FIG. 13 shows a western blot of recombinant TAP-tagged methyl coenzyme M reductase (MCR) and MmpX in M. maripaludis following growth with high (800 μM) or low phosphate (40 μM or 80 μM). The TAP tag comprised (strep)₂-(FLAG)₃, where the strep tag was used for purification and the FLAG tag was used for Western blotting. The μg of protein loaded is given below the phosphate concentration. PC, positive control of N-terminal FLAG-BAP™ (Sigma Aldrich) fusion protein.

FIGS. 14A-14C show production of MCR with Ppst. FIG. 14A map of the shuttle vector for expression of the mcr operon regulated by the 93-bp Ppst. The plasmid also contains ColE1 ori, an origin of replication in E. coli; ORFLESS1, a possible origin of replication in Methanococcus; RBS, ribosome binding site; Ts terminator, a synthetic terminator; Pmcr, mcr promoter from M. voltae; pur, a puromycin resistance cassette for positive selection in M. maripaludis; Tmcr, mcr terminator from M. voltae; amp, the ampicillin resistance cassette for positive selection in E. coli. In the control plasmid, Ppst was replaced with the constitutive PhmvA. FIG. 14B comparison of expression levels of MCRₐₑₒ regulated by PhmvA versus Ppst. The M. maripaludis host cells were cultivated with the formate medium containing 80 μM initial Pi. Protein levels were determined by Western blotting. Levels of expression in % of total protein is given above each bar. FIG. 14C SDS-PAGE analysis of the purified MCRₐₑₒ demonstrating the presence of all three subunits. The molecular weight (MW) of the ladder is indicated on the left. The protein bands marked with arrows were excised, and the protein identities were confirmed by LC-MS/MS.

### DETAILED DESCRIPTION OF THE INVENTION

Many challenges are involved in the synthetic conversion of CO₂ into CH₄ and the production of valuable products from CH₄. These include catalyst stability and selectivity, the cost of catalysts and associated technology, and the need to avoid contamination.

Methanogenic archaea are a potentially useful microbial cell factory to produce proteins, biocatalysts and biochemicals through recombinant gene expression. However, the use of methanogenic archaea in a microbial cell factory has been hindered by a lack of molecular tools for auto-inducible gene expression. A regulatory system that uncouples growth from recombinant gene expression is required, especially when the product of interest or its precursors are toxic, or when the engineered pathway competes with endogenous pathways that are essential for cell growth.

Methanococcus maripaludis is a rapidly-growing mesophilic methanogen that utilizes H₂:CO₂ or formate as its sole energy and carbon source for biomass and methane formation and is a promising candidate for a microbial cell factory. A tetracycline inducible expression system developed for regulated gene expression in Methanosarcina cells has been trialled in M. maripaludis. However, it is not suitable for large-scale (e.g. industrial) cultures, due to the high cost of tetracycline. Of the two remaining regulatory systems known in M. maripaludis, one is nitrogen-dependent and the other is temperature-dependent. However, regulation in both systems is accompanied by other large changes in gene expression due to general responses to nitrogen limitation and temperature. Moreover, to switch gene expression on and off, the nitrogen-dependent promoter needs a laborious change in the nitrogen source between ammonia and alanine or dinitrogen, while the temperature-dependent promoter requires a dramatic change in temperature. Thus, these systems are not practical for large scale (e.g., industrial) cultures.

The present inventors have now identified a region of the M. maripaludis genome containing a phosphate transporter gene promoter (Ppst) and have introduced the promoter into a shuttle vector, thereby providing a recombinant gene expression system. This system induces recombinant gene expression in response to limiting the phosphate levels in a cell culture (without the need for an external inducer such as tetracycline).

When the concentration of phosphate is high, cells grow and recombinant gene expression is low. When the concentration of phosphate drops to limiting concentration during growth, recombinant gene expression can be upregulated at the same time as biomass production is limited. In this way, growth is decoupled from expression, allowing the production of proteins or metabolites inhibitory to growth.

Apart from manipulating the concentrations of phosphate and other growth conditions, protein expression can be controlled by modifying the transcription and/or translation initiation rates, for example by altering the core promoter elements, 5′ untranslated region (UTR), ribosome binding sites (RBS), and/or start codon.

This provides a practical regulatory system for protein overexpression and is particularly suitable for large-scale (e.g. industrial) cultures and thus for large-scale protein and chemical production.

### Definitions

Unless defined otherwise, all technical and scientific terms used herein have the same meaning as commonly understood by one of ordinary skill in the art to which this invention belongs. In case of conflict, the present application, including the definitions, will prevail.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

To facilitate an understanding of the present invention, a number of terms and phrases are defined below.

As used in the present disclosure and claims, the singular forms “a,” “an,” and “the” include plural forms unless the context clearly dictates otherwise.

Wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include “A and B”, “A or B”, “A”, and “B”.

The terms “cell” (e.g., host cell) and “cell culture” include the primary subject cells and any progeny thereof, without regard to the number of transfers. It should be understood that not all progeny are exactly identical to the parental cell (due to deliberate or inadvertent mutations or differences in environment). However, such altered progeny are included in these terms, so long as the progeny retain the same functionality as that of the originally transformed cell.

The term “gene” is used broadly to refer to any segment of nucleic acid molecule (typically DNA, but optionally RNA) that encodes a protein or that can be transcribed into a functional RNA. Genes may include sequences that are transcribed but are not part of a final, mature, and/or functional RNA transcript, and genes that encode proteins may further comprise sequences that are transcribed but not translated, for example, 5′ untranslated regions, 3′ untranslated regions, introns, etc. Further, genes may optionally comprise regulatory sequences required for their expression, and such sequences may be, e.g., sequences that are not transcribed or translated. Genes can be obtained from a variety of sources, including cloning from a source of interest or synthesizing from known or predicted sequence information, and may include sequences designed to have desired parameters.

The term “nucleic acid” or “nucleic acid molecule” refers to, e.g., DNA or RNA (e.g., mRNA). The nucleic acid molecules can be double-stranded or single-stranded; single stranded RNA or DNA can be the coding (sense) strand or the non-coding (antisense) strand.

The term “isolated” nucleic acid, such as an isolated protein or nucleic acid as used herein, refers to a biomolecule removed from the context in which the biomolecule exists in nature. An isolated biomolecule can be, in some instances, partially or substantially purified. For example, an isolated nucleic acid molecule can be a nucleic acid sequence that has been excised from the chromosome, genome, or episome into which it is integrated in nature.

A “purified” nucleic acid molecule or nucleotide sequence, or protein or polypeptide sequence, is substantially free of cellular material and cellular components. The purified nucleic acid molecule or protein may be free of chemicals beyond buffer or solvent, for example. “Substantially free” is not intended to mean that other components beyond the novel nucleic acid molecules are undetectable.

“Exogenous nucleic acid molecule” or “exogenous gene” refers to a nucleic acid molecule or gene that has been introduced (“transformed”) into a cell. A transformed cell may be referred to as a recombinant cell, into which additional exogenous gene(s) may be introduced. A descendent of a cell transformed with a nucleic acid molecule is also referred to as “transformed” if it has inherited the exogenous nucleic acid molecule. The exogenous gene may be from a different species (and so “heterologous”), or from the same species (and so “homologous”), relative to the cell being transformed. An “endogenous” nucleic acid molecule, gene or protein is a native nucleic acid molecule, gene or protein as it occurs in, or is naturally produced by, the host.

The term “heterologous” when used in reference to a polynucleotide, a gene, a nucleic acid, a polypeptide, or an enzyme refers to a polynucleotide, gene, a nucleic acid, polypeptide, or an enzyme not derived from the host species. For example, “heterologous gene” or “heterologous nucleic acid sequence” as used herein, refers to a gene or nucleic acid sequence from a different species than the species of the host organism it is introduced into. When referring to a gene regulatory sequence or to an auxiliary nucleic acid sequence used for maintaining or manipulating a gene sequence (e.g. a 5′ untranslated region, 3′ untranslated region, poly A addition sequence, intron sequence, splice site, ribosome binding site, internal ribosome entry sequence, genome homology region, recombination site, etc.) or to a nucleic acid sequence encoding a protein domain or protein localization sequence, “heterologous” means that the regulatory or auxiliary sequence or sequence encoding a protein domain or localization sequence is from a different source than the gene with which the regulatory or auxiliary nucleic acid sequence or nucleic acid sequence encoding a protein domain or localization sequence is juxtaposed in a construct, genome, chromosome or episome. Thus, a promoter operably linked to a gene to which it is not operably linked to in its natural state may be referred to herein as a “heterologous promoter,” even though the promoter may be derived from the same species as the gene to which it is linked. Similarly, when referring to a protein localization sequence or protein domain of an engineered protein, “heterologous” means that the localization sequence or protein domain is derived from a protein different from that into which it is incorporated by genetic engineering.

The term “recombinant” or “engineered” nucleic acid molecule as used herein, refers to a nucleic acid molecule that has been altered through human intervention. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes cojoined nucleotide sequences that are not cojoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector.

The term “recombinant protein” as used herein refers to a protein produced by genetic engineering. The terms “peptide,” “polypeptide” and “protein” are used interchangeably herein, although “peptide” may be used to refer to a polypeptide having no more than about 100 amino acids, or no more than about 60 amino acids.

When applied to organisms, the terms “transgenic” or “recombinant” or “engineered” or “genetically engineered” refer to organisms that have been manipulated by introduction of an exogenous or recombinant nucleic acid sequence into the organism. Non-limiting examples of such manipulations include gene knockouts, targeted mutations and gene replacement, promoter replacement, deletion, or insertion, as well as introduction of transgenes into the organism. For example, a transgenic microorganism can include an introduced exogenous regulatory sequence, for example a promoter sequence, operably linked to an endogenous gene of the transgenic microorganism. Recombinant or genetically engineered organisms can also be organisms into which constructs for gene “knock down” have been introduced. Such constructs include, but are not limited to, RNAi, microRNA, shRNA, antisense, and ribozyme constructs. Also included are organisms whose genomes have been altered by the activity of meganucleases or zinc finger nucleases. A heterologous or recombinant nucleic acid molecule can be integrated into a genetically engineered/recombinant organism's genome or, in other instances, not integrated into a recombinant/genetically engineered organism's genome. As used herein, “recombinant microorganism” or “recombinant host cell” includes progeny or derivatives of the recombinant microorganisms of the invention. Because certain modifications may occur in succeeding generations from either mutation or environmental influences, such progeny or derivatives may not, in fact, be identical to the parent cell, but are still included within the scope of the term as used herein.

The term “expression cassette” as used herein, refers to a nucleic acid construct that encodes a protein or functional RNA (e.g. a tRNA, a short hairpin RNA, one or more microRNAs, a ribosomal RNA, etc.) operably linked to expression control elements, such as a promoter, and optionally, any or a combination of other nucleic acid sequences that affect the transcription or translation of the gene, such as, but not limited to, a transcriptional terminator, a ribosome binding site, a splice site or splicing recognition sequence, an intron, an enhancer, a polyadenylation signal, an internal ribosome entry site, etc.

“Regulatory sequence”, “regulatory element”, or “regulatory element sequence” refers to a nucleotide sequence located upstream (5′), within, or downstream (3′) of a coding sequence or functional RNA-encoding sequence. Transcription of the coding sequence or functional RNA-encoding sequence and/or translation of an RNA molecule resulting from transcription of the coding sequence are typically affected by the presence or absence of the regulatory sequence. These regulatory element sequences may comprise promoters, cis-elements, enhancers, terminators, or introns. Regulatory elements may be isolated or identified from untranslated regions (UTRs) from a particular polynucleotide sequence. Any of the regulatory elements described herein may be present in a chimeric or hybrid regulatory expression element. Any of the regulatory elements described herein may be present in a recombinant construct of the present invention.

The terms “promoter”, “promoter region”, or “promoter sequence” refer to a nucleic acid sequence capable of binding RNA polymerase to initiate transcription of a gene in a 5′ to 3′ (“downstream”) direction. A gene is “under the control of” or “regulated by” a promoter when the binding of RNA polymerase to the promoter is the proximate cause of said gene's transcription. The promoter or promoter region typically provides a recognition site for RNA polymerase and other factors necessary for proper initiation of transcription. A promoter may be isolated from the 5′ untranslated region (5′ UTR) of a genomic copy of a gene. Alternatively, a promoter may be synthetically produced or designed by altering known DNA elements. Also considered are chimeric promoters that combine sequences of one promoter with sequences of another promoter. Promoters may be defined by their expression pattern based on, for example, metabolic, environmental, or developmental conditions. A promoter can be used as a regulatory element for modulating expression of an operably linked transcribable polynucleotide molecule, e.g., a coding sequence or functional RNA sequence. Promoters may contain, in addition to sequences recognized by RNA polymerase and, preferably, other transcription factors, regulatory sequence elements such as cis-elements or enhancer domains that affect the transcription of operably linked genes.

The term “inducible” promoter refers to a promoter having activity dependent on environmental and developmental conditions. The activity of an inducible promoter is dependent on the external environment, such as light and culture medium composition. In some examples, an inducible promoter is inactive in the presence of one or more nutrients (e.g., inorganic phosphates) but active when the one or more nutrients is/are absent. Thus, an inducible promoter is a promoter that is active in response to particular environmental conditions, such as the presence or absence of a nutrient or regulator, the presence or absence of light, etc. In contrast, a “constitutive” promoter is a promoter that is active under most environmental and developmental conditions.

The term “operably linked,” as used herein, denotes a configuration in which a control sequence or localization sequence is placed at an appropriate position relative to a sequence that encodes a polypeptide or functional RNA such that the control sequence directs or regulates the expression or localization of the mRNA encoding the polypeptide, the polypeptide, and/or the functional RNA. Thus, a promoter is in operable linkage with a nucleic acid sequence if it can mediate transcription of the nucleic acid sequence. When introduced into a host cell, an expression cassette that includes a control sequence can result in transcription of the gene to which it is operably linked and/or translation of an encoded RNA or polypeptide under appropriate conditions. Antisense or sense constructs that are not or cannot be translated are not excluded by this definition. In the case of both expression of transgenes and suppression of endogenous genes (e.g., by antisense, or sense suppression) one of ordinary skill will recognize that the inserted polynucleotide sequence need not be identical, but may be only substantially identical to a sequence of the gene from which it was derived. As explained herein, these substantially identical variants are specifically covered by reference to a specific nucleic acid sequence.

The term “selectable marker” or “selectable marker gene” as used herein includes any gene that confers a phenotype on a cell in which it is expressed to facilitate the selection of cells that are transfected or transformed with a nucleic acid construct of the invention. The term may also be used to refer to gene products that effectuate said phenotypes.

A “reporter gene” is a gene encoding a protein that is detectable or has an activity that produces a detectable product. A reporter gene can encode a visual marker or enzyme that produces a detectable signal. Non-limiting examples include: a β-glucuronidase gene, a β-galactosidase gene, or a gene encoding a fluorescent protein, including but not limited to a blue, cyan, green, red, or yellow fluorescent protein, a photoconvertible, photoswitchable, or optical highlighter fluorescent protein, or any of variant thereof, including, without limitation, codon-optimized, rapidly folding, monomeric, increased stability, and enhanced fluorescence variants.

The term “transformation” as used herein refers to the introduction of one or more exogenous nucleic acid sequences or polynucleotides into a host cell or organism by using one or more physical, chemical, or biological methods. Physical and chemical methods of transformation (i.e., “transfection”) include, by way of non-limiting example, polyethylene glycol (PEG) mediated transformation. Biological methods of transformation include, by way of non-limiting example, transfer of DNA using engineered viruses or microbes (e.g., E. coli).

The terms, “identical” or percent “identity”, in the context of two or more nucleic acids or polypeptide sequences, refer to two or more sequences or subsequences that are the same or have a specified percentage of amino acid residues or nucleotides that are the same, when compared and aligned for maximum correspondence over a comparison window. The degree of amino acid or nucleic acid sequence identity can be determined by various computer programs for aligning the sequences to be compared based on designated program parameters. For example, sequences can be aligned and compared using the local homology algorithm of Smith & Waterman (1981) Adv. Appl. Math. 2:482-89, the homology alignment algorithm of Needleman & Wunsch (1970) J. Mol. Biol. 48:443-53, or the search for similarity method of Pearson & Lipman (1988) Proc. Nat'l. Acad. Sci. USA 85:2444-48, and can be aligned and compared based on visual inspection or can use computer programs for the analysis (for example, GAP, BESTFIT, FASTA, and TFASTA in the Wisconsin Genetics Software Package, Genetics Computer Group, 575 Science Dr., Madison, WI).

The BLAST algorithm, described in Altschul et al. (1990) J. Mol. Biol. 215:403-10, is publicly available through software provided by the National Center for Biotechnology Information (available at http://www.ncbi.nlm.nih.gov). This algorithm identifies high scoring sequence pairs (HSPS) by identifying short words of length W in the query sequence, which either match or satisfy some positive-valued threshold score T when aligned with a word of the same length in a database sequence. T is referred to as the neighborhood word score threshold (Altschul et al., 1990). Initial neighborhood word hits act as seeds for initiating searches to find longer HSPs containing them. The word hits are then extended in both directions along each sequence for as far as the cumulative alignment score can be increased. Cumulative scores are calculated for nucleotides sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments; or the end of either sequence is reached. For determining the percent identity of a nucleic acid sequence, the default parameters of the BLAST programs can be used. For analysis of nucleic acid sequences, the BLASTN program defaults are word length (W), 11; expectation (E), 10; M=5; N=−4; and a comparison of both strands. The TBLASTN program (using a protein sequence to query nucleotide sequence databases) uses as defaults a word length (W) of 3, an expectation (E) of 10, and a BLOSUM 62 scoring matrix. See Henikoff & Henikoff (1989) Proc. Nat'l. Acad. Sci. USA 89:10915-19.

In addition to calculating percent sequence identity, the BLAST algorithm also performs a statistical analysis of the similarity between two sequences (see, e.g., Karlin & Altschul (1993) Proc. Nat'l. Acad. Sci. USA 90:5873-87). The smallest sum probability (P(N)), provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, preferably less than about 0.01, and more preferably less than about 0.001.

### Nucleotide Sequences

Nucleotide sequences were identified and isolated, which find use as promoter sequences (e.g., inducible or auto-inducible promoter sequences) in the expression of genes (e.g., genes encoding enzymes associated with methanogenesis or reverse methanogenesis) in prokaryotic microorganisms (e.g., methanogenic archaea). The methods by which these sequences were identified is described more fully in the Examples set forth herein.

Thus, an isolated DNA molecule is provided, comprising a sequence having at least about 80% identity to at least about 80 contiguous nucleotides of SEQ ID No. 1.

For example, the isolated DNA molecule can comprise a sequence that can have at least about 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 80 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 81 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 82 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 83 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 84 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 85 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 86 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 87 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 88 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 89 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 90 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 91 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 92 contiguous nucleotides of SEQ ID No. 1. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to about 93 contiguous nucleotides of SEQ ID No. 1.

Also provided herein is an isolated DNA molecule comprising a sequence having at least about 80% identity to at least about 200 contiguous nucleotides of SEQ ID No. 2.

For example, the isolated DNA molecule can comprise a sequence that can have at least about 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 200 contiguous nucleotides of SEQ ID No. 2. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 205 contiguous nucleotides of SEQ ID No. 2. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 210 contiguous nucleotides of SEQ ID No. 2. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 215 contiguous nucleotides of SEQ ID No. 2. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 220 contiguous nucleotides of SEQ ID No. 2. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 225 contiguous nucleotides of SEQ ID No. 2. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 230 contiguous nucleotides of SEQ ID No. 2. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 235 contiguous nucleotides of SEQ ID No. 2. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to at least about 240 contiguous nucleotides of SEQ ID No. 2. The isolated DNA molecule can comprise a sequence that can have at least about 80%, 81%, 82%, 83%, 84%, 85%, 86%, 87%, 88%, 89%, 90%, 91%, 92%, 93%, 94%, 95%, 96%, 97%, 98% or 99% identity, or that can have about 100% percent identity, to about 243 contiguous nucleotides of SEQ ID No. 2.

The sequences provided herein (e.g., a sequence having a percent identity to a number of contiguous nucleotides of SEQ ID No. 1 or a sequence having a percent identity to a number of contiguous nucleotides of SEQ ID No. 2) can comprise a region, for example a region rich in A nucleotides and T nucleotides (“an AT-rich region”) having at least about 80%, 81%, 82%, 83%, 84%, 85%, 86%, 87%, 88%, 89%, 90%, 91%, 92%, 93%, 94%, 95%, 96%, 97%, 98%, or 99% identity, or having about 100% identity, to at least about 15, 16, 17, 18, 19, 20, 21, 22, 23 or 24, or to about 25, contiguous nucleotides of SEQ ID No. 3. For example, the region may be a region having about 80% identity to at least about 20 contiguous nucleotides of SEQ ID No. 3.

The sequences provided herein (e.g., a sequence having a percent identity to a number of contiguous nucleotides of SEQ ID No. 1 or a sequence having a percent identity to a number of contiguous nucleotides of SEQ ID No. 2) can comprise a region, for example an untranslated region, having at least about 80%, 81%, 82%, 83%, 84%, 85%, 86%, 87%, 88%, 89%, 90%, 91%, 92%, 93%, 94%, 95%, 96%, 97%, 98%, or 99% identity, or having about 100% identity, to at least about 15, 16, 17, 18, 19, 20, or 21, or to about 22, contiguous nucleotides of SEQ ID No. 4. For example, the region may be a region having about 80% identity to at least about 18 contiguous nucleotides of SEQ ID No. 4. Optionally, both a region having a percent identity to a number of contiguous nucleotides of SEQ ID No. 3 and a region having a percent identity to a number of contiguous nucleotides of SEQ ID No. 4 can be present in a sequence provided herein, e.g., in a sequence having a percent identity to a number of contiguous nucleotides of SEQ ID No. 1, or in a sequence having a percent identity to a number of contiguous nucleotides of SEQ ID No. 2.

The sequences provided herein (e.g., a sequence having a percent identity to a number of contiguous nucleotides of SEQ ID No. 1 or a sequence having a percent identity to a number of contiguous nucleotides of SEQ ID No. 2) can comprise a region, for example an untranslated region, having at least about 80%, 81%, 82%, 83%, 84%, 85%, 86%, 87%, 88%, 89%, 90%, 91%, 92%, 93%, 94%, 95%, 96%, 97%, 98%, or 99% identity, or having about 100% identity, to at least about 15, 16, 17, 18, 19, or 20, or to about 21, contiguous nucleotides of SEQ ID No. 5. For example, the region may be a region having about 80% identity to at least about 17 contiguous nucleotides of SEQ ID No. 5. Optionally, both a region having a percent identity to a number of contiguous nucleotides of SEQ ID No. 3 and a region having a percent identity to a number of contiguous nucleotides of SEQ ID No. 5 can be present in a sequence provided herein, e.g., in a sequence having a percent identity to a number of contiguous nucleotides of SEQ ID No. 1, or in a sequence having a percent identity to a number of contiguous nucleotides of SEQ ID No. 2.

The sequences provided herein (e.g., a sequence having a percent identity to a number of contiguous nucleotides of SEQ ID No. 1 or a sequence having a percent identity to a number of contiguous nucleotides of SEQ ID No. 2) can comprise a region, for example an untranslated region, having at least about 80%, 81%, 82%, 83%, 84%, 85%, 86%, 87%, 88%, 89%, 90%, 91%, 92%, 93%, 94%, 95%, 96%, 97%, 98%, or 99% identity, or having about 100% identity, to at least about 15, 16, 17, 18, or 19, or to about 20, contiguous nucleotides of SEQ ID No. 6. For example, the region may be a region having about 80% identity to at least about 16 contiguous nucleotides of SEQ ID No. 6. Optionally, both a region having a percent identity to a number of contiguous nucleotides of SEQ ID No. 3 and a region having a percent identity to a number of contiguous nucleotides of SEQ ID No. 6 can be present in a sequence provided herein, e.g., in a sequence having a percent identity to a number of contiguous nucleotides of SEQ ID No. 1, or in a sequence having a percent identity to a number of contiguous nucleotides of SEQ ID No. 2.

The sequences provided herein may optionally have a total length of no more than 100, 200, 300, 400, 500, 600, 700, 800, 900 or 1000 base pairs (bp). The sequences provided herein may optionally have a total length of no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 kilobases (kb). The sequences provided herein may optionally have a total length of no more than 1, 2, 3, 4, 5, 6, 7, 8, 9 or 10 megabases (Mb).

### Promoters.

An isolated DNA molecule provided herein can find use, for example, as a sequence that when operably linked to a nucleic acid sequence can affect expression of the nucleic acid sequence, which can comprise, for example, a sequence encoding a polypeptide or functional RNA. For example, the isolated DNA molecule may mediate transcription of the operably-linked nucleic acid sequence (or a portion thereof) as a promoter. Thus, isolated DNA molecules provided herein (e.g., a sequence having a percent identity to a number of contiguous nucleotides of SEQ ID No. 1 or a sequence having a percent identity to a number of contiguous nucleotides of SEQ ID No. 2) can have promoter activity. Thus, provided herein are promoters that can comprise or consist of one or more (e.g., 2, 3, 4 or 5) isolated DNA molecules provided herein.

Methods for assessing the functionality of nucleotide sequences for promoter activity, as well as for enhancing or decreasing the activity of proximal promoters, are well-known in the art. For example, promoter function can be validated by confirming the ability of the putative promoter (or promoter variant or fragment) to drive expression of a selectable marker gene conferring resistance to an antibiotic or antibiotics (e.g., puromycin, neomycin, 8-azahypoxanthine, 6-azauracil) to which the putative promoter (or promoter fragment or variant) is operably linked, by detecting and, optionally, analyzing, resistant colonies after plating of cells transformed with the promoter construct on selective media.

Additionally or alternatively, promoter activity may be assessed by measuring the levels of RNA transcripts produced from a promoter construct, for example, using reverse transcription-polymerase chain reaction (RT-PCR), by detection of the expressed protein, or by in vivo assays that rely on an activity of the protein encoded by the transcribed sequence. By way of non-limiting example, promoter activity can be assessed by in vivo assays using a fluorescent protein gene to determine the functionality of any of the sequences disclosed herein, including sequences of reduced size or having one or more nucleotide changes with respect to any of the sequences disclosed herein, such as SEQ ID Nos. 1 or 2.

Thus, provided herein is a promoter comprising a sequence provided herein, such as a sequence having a percent identity to a number of contiguous nucleotides of SEQ ID No. 1, as described hereinabove. For example, provided herein is a promoter comprising a sequence having at least about 80% identity to at least about 80 contiguous nucleotides of SEQ ID No. 1. Also provided herein is a promoter comprising a sequence having a percent identity to a number of contiguous nucleotides of SEQ ID No. 2, as described hereinabove. For example, provided herein is a promoter comprising a sequence having at least about 80% identity to at least about 200 contiguous nucleotides of SEQ ID No. 2.

A promoter provided herein may optionally comprise a sequence including a region having a percent identity to a number of contiguous nucleotides of SEQ ID No. 3, as described hereinabove, for example about 80% identity to at least about 20 contiguous nucleotides of SEQ ID No. 3. A promoter provided herein may optionally comprise a sequence including a region having a percent identity to a number of contiguous nucleotides of SEQ ID No. 4, SEQ ID No. 5, or SEQ ID No. 6, as described hereinabove. For example, the region may be: a region having about 80% identity to at least about 18 contiguous nucleotides of SEQ ID No. 4; a region having about 80% identity to at least about 17 contiguous nucleotides of SEQ ID No. 5; or a region having about 80% identity to at least about 16 contiguous nucleotides of SEQ ID No. 6. A promoter provided herein may optionally comprise a sequence including both: a region having a percent identity to a number of contiguous nucleotides of SEQ ID No. 4, SEQ ID No. 5, or SEQ ID No. 6; and a region having a percent identity to a number of contiguous nucleotides of SEQ ID No. 3.

A promoter provided herein can optionally be an inducible promoter (e.g., an inducible phosphate responsive promoter, such as an inducible inorganic phosphate responsive promoter). For example, the promoter can be active in culture conditions in which one or more nutrients (e.g., inorganic phosphates) are deficient, but not in culture conditions in which the one or more nutrients are sufficient for proliferation and/or growth of the culture. For example, a promoter provided herein can optionally direct expression of an operably linked nucleic acid sequence under conditions in which a host cell that includes the promoter construct is limited in inorganic phosphate availability (inorganic phosphate depletion/deficiency) but not under conditions in which a host cell that includes the promoter construct has unlimited inorganic phosphate availability (inorganic phosphate replete conditions). A promoter provided herein can optionally be an autoinducible promoter (e.g., an autoinducible phosphate responsive promoter, such as an autoinducible inorganic phosphate responsive promoter).

Without wishing to be bound by theory, promoters allow RNA polymerase to attach to DNA near a gene in order for transcription to take place. Promoters contain specific DNA sequences that provide transcription factors to an initial binding site from which they can recruit RNA polymerase binding. These transcription factors have specific protein motifs that enable them to interact with specific corresponding nucleotide sequences to regulate gene expressions.

A proximal promoter sequence may optionally be approximately 250 basepairs (bp) upstream of the translational start site of the open reading frame of the gene and may contain, in addition to sequences for binding RNA polymerase, specific transcription factor binding sites. Some promoters also include a distal sequence upstream of the gene that may contain additional regulatory elements, often with a weaker influence than the proximal promoter. Further, promoters or portions of promoters provided herein may optionally be combined in series to achieve a stronger level of expression or a more complex pattern of regulation.

Thus, a promoter provided herein can optionally comprise more than one, such as 2, 3, 4 or 5, of the sequences provided herein. For example, a promoter provided herein can comprise more than one sequence (e.g., 2, 3, 4 or 5 sequences) having a percent identity to a number of contiguous nucleotides of SEQ ID No. 1, such as about 80% identity to at least about 80 contiguous nucleotides of SEQ ID No. 1. A promoter provided herein can optionally comprise more than one sequence (e.g., 2, 3, 4 or 5 sequences) having a percent identity to a number of contiguous nucleotides of SEQ ID No. 2, such as about 80% identity to at least about 200 contiguous nucleotides of SEQ ID No. 2. A promoter provided herein can optionally comprise one or more sequences (e.g., 1, 2 or 3 sequences) having a percent identity to a number of contiguous nucleotides of SEQ ID No 1 (such as about 80% identity to at least about 80 contiguous nucleotides of SEQ ID No. 1) in addition to one or more sequences (e.g., 1, 2 or 3 sequences) having a percent identity to a number of contiguous nucleotides of SEQ ID No. 2 (such as about 80% identity to at least about 200 contiguous nucleotides of SEQ ID No. 2).

A promoter provided herein can optionally comprise further elements in addition to one or more sequences provided herein. Additional regulatory element sequences contemplated herein for use with the promoters provided herein include cis regulatory elements (e.g., further promoters, enhancers, silencers, operators) and trans regulatory elements. Regulatory elements may be isolated or identified from untranslated regions (UTRs) from a particular polynucleotide sequence. Any of the regulatory elements described herein may optionally be present in a chimeric or hybrid regulatory expression element.

A promoter or promoter region can optionally include variants of the promoters provided herein, derived by deleting sequences, duplicating sequences, or adding sequences from other promoters or as designed, for example, by bioinformatics, or by subjecting the promoter to random or site-directed mutagenesis, etc.

In various optional examples, a promoter provided herein can mediate transcription of an operably linked nucleic acid sequence in a prokaryotic cell (e.g., an archaeal cell, such as a methanogenic archaeal cell, such as a Methanococcus maripaludis cell). In some instances, a promoter provided herein can mediate transcription of an operably linked nucleic acid sequence in a prokaryotic cell, during culturing of the cell under conditions of inorganic phosphate depletion, but not during culturing of the cell under inorganic phosphate replete conditions. For example, a promoter provided herein can mediate transcription of an operably linked nucleic acid sequence in a methanogenic archaeal cell (e.g., a Methanococcus maripaludis cell) cultured under conditions of inorganic phosphate depletion, but not under inorganic phosphate replete conditions.

The activity or strength of a promoter may be measured in terms of the amount of RNA it produces, or the amount of protein accumulation in a cell or tissue, which can optionally be measured by an activity of the expressed protein, e.g., fluorescence, luminescence, acyltransferase activity, etc., relative to a promoter whose transcriptional activity has been previously assessed, relative to a promoterless construct, or relative to non-transformed cells. For example, the activity or strength of a promoter may be measured in terms of the amount of mRNA accumulated that corresponds to a nucleic acid sequence to which it is operably linked in a cell, relative to the total amount of mRNA or protein produced by the cell. The promoter preferably expresses an operably linked nucleic acid sequence at a level greater than 0.01%; preferably in a range of about 0.5% to about 20% (w/w) of the total cellular RNA. The activity can also be measured by quantifying fluorescence, luminescence, or absorbance of the cells or a product made by the cells or an extract thereof, depending on the activity of a reporter protein that may be expressed from the promoter. The activity or strength of a promoter may be expressed relative to a well-characterized promoter (for which transcriptional activity was previously assessed). For example, a less-characterized promoter may optionally be operably linked to a reporter sequence (e.g., a fluorescent protein) and introduced into a specific cell type. A well-characterized promoter is similarly prepared and introduced into the same cellular context. Transcriptional activity of the unknown promoter is determined by comparing the amount of reporter expression, relative to the well characterized promoter.

Promoter activity can optionally be assessed by measuring the amount, relative to total protein in cell-free extracts, of a recombinant protein made by the cells. The promoter (e.g., an inducible or autoinducible phosphate responsive promoter, such as an inducible or autoinducible inorganic phosphate responsive promoter) may express a recombinant protein (e.g., a recombinant enzyme, such as an enzyme associated with methanogenesis or reverse methanogenesis) at a level greater than 1%, e.g., in a range of about 1% to about 20% (w/w), about 1% to about 15%, or about 1% to about 10% (w/w) of total protein in cell-free extracts.

### Expression Cassettes

Expression cassettes are also provided herein. The expression cassettes comprise a promoter comprising an isolated DNA molecule as provided herein, and a gene encoding a polypeptide or a functional RNA sequence, wherein the gene is operably linked to the promoter. The gene may optionally be positioned downstream of the promoter sequence. Optionally, the expression cassette also comprises one or more additional regulatory elements as described herein. The basic techniques for operably linking two or more sequences of DNA together are familiar to the person of ordinary skill in the art.

The promoters of the invention can optionally be used with any exogenous or endogenous gene(s). An exogenous or endogenous gene contemplated herein may encode a protein or a polypeptide. For example, the gene can be an exogenous or endogenous gene that encodes an enzyme that catalyses one or more reactions in methanogenesis or in reverse methanogenesis (e.g., methylcoenzyme M reductase). Alternatively, the gene can encode a functional RNA. Any known or later-discovered exogenous or endogenous gene which encodes a desired product can be operably linked to a promoter sequence provided herein using known methods.

Non-limiting examples of known genes suitable for use with the promoters provided herein include genes encoding enzymes associated with methanogenesis or reverse methanogenesis, such as: formylmethanofuran dehydrogenase (Fmd); formylmethanofuran:H₄MPT formyltransferase (Ftr); methenyl-H₄MPT cyclohydrolase (Mch); F₄₂₀-dependent methenyl-H₄MPT cyclohydrolase (Mtd); H₂-forming (F₄₂₀-independent) methenyl-H₄MPT cyclohydrolase (Hmd); methylene-H₄MPT reductase (Mer); methyl-H₄MPT:coenzyme M methyltransferase (Mtr); methyl-coenzyme M reductase (Mcr); heterodisulfide reductase (Hdr); and F₄₂₀-reducing hydrogenase (Frh).

Further non-limiting examples of known genes suitable for use with the promoters provided herein include reporter proteins (e.g., fluorescent proteins or enzymes that produce detectable products).

In further examples, an expression cassette can comprise a promoter provided herein operably linked to a gene encoding a functional RNA, optionally wherein the functional RNA is an antisense RNA, a small hairpin RNA, a microRNA, an siRNA, an snoRNA, a piRNA, or a ribozyme.

### Vectors

Also provided herein are vectors that comprise the expression cassettes provided herein. The vector can be a plasmid. The vector can be a shuttle vector, such as a pURB500 shuttle vector.

A vector provided herein may optionally further comprise one or more selectable markers and/or reporter genes. For example, in addition to the expression cassette provided herein, the vector may optionally further comprise one or more of: a selectable marker gene, a reporter gene, an origin of replication, and one or more sequences for promoting integration of the expression cassette into the host genome.

By way of example, a vector that includes an expression cassette may optionally include, as one or more selectable markers, one or more genes conferring resistance to an antibiotic or antibiotics (e.g., puromycin, neomycin, 8-azahypoxanthine, 6-azauracil) so that transformants can be selected by exposing the cells to the agent(s) and selecting those cells which survive the encounter. The selectable marker can optionally be operably linked to and/or under the control of a promoter. The promoter regulating expression of the selectable marker may be inducible (e.g. autoinducible) and can be, for example, any promoter provided herein, or another promoter. The selectable marker may optionally be placed under the control of the expression cassette promoter.

If a vector provided herein that includes an expression cassette lacks a selectable marker gene, transformants may be selected by routine methods familiar to those skilled in the art, such as, by way of a non-limiting example, extracting nucleic acid from the putative transformants and screening by PCR.

Alternatively or in addition, transformants may be screened by detecting expression of a reporter gene, such as but not limited to a gene encoding a fluorescent protein, such as any of the blue, cyan, green, red, yellow, photoconvertible, or photoswitchable fluorescent proteins or any of their variants. The reporter gene can be operably linked to and/or under the control of a promoter. The promoter regulating expression of the reporter gene may be inducible and can be, for example, any promoter provided herein, or another promoter. The reporter gene may optionally be placed under the control of the expression cassette promoter.

In addition to the promoters provided herein, one skilled in the art would know various promoters, introns, enhancers, promoter proximal DNA element, initiator element, transit peptides, targeting signal sequences, 5′ and 3′ untranslated regions (UTRs), IRES, 2A sequences, and terminator sequences, as well as other molecules involved in the regulation of gene expression that are useful in the design of effective expression vectors. The expression vector may contain one or more enhancer elements. Enhancers are short regions of DNA that can bind trans-acting factors to enhance transcription levels. Although enhancers usually act in cis, an enhancer need not be particularly close to its target gene. Enhancers can sometimes be located in introns.

The vector can further include one or more additional genes or constructs for transfer into the host cell, which can optionally be operably linked to a promoter provided herein, or can optionally be operably linked to another promoter.

### Cells

A prokaryotic host cell is also provided, transformed with an expression vector as provided herein. Thus, a prokaryotic host cell is provided comprising a vector as described herein. A prokaryotic host cell is provided comprising an isolated nucleic acid molecule as described herein. A prokaryotic host cell is provided comprising an expression cassette as described herein.

The prokaryotic host cell can be an archaeal cell. Archaea are single-celled microorganisms. Although their cellular structure may be similar to that of bacteria, they form a separate domain of life and are evolutionarily distinct from bacteria and eukaryotes. Methanogenic archaea are obligate anaerobes. Typically, methanogenic archaea are found in oxygen-depleted environments.

Archaeal transcription is different from that of bacteria. The archaeal basal transcription apparatus closely resembles that of eukaryotes. Archaeal RNA polymerase (RNAP) requires the activity of transcription initiation factors, including the TATA-box binding protein (TBP) and transcription factor B (TFB) to recognize promoter sequences, and these initiation factors are homologs to their eukaryotic counterparts. Transcription initiation occurs upon the sequence-specific binding of TBP to a TATA box upstream of the transcription start sites (TSS) and TFB to a factor B recognition element (BRE) upstream of the TATA box. RNAP is then recruited to the TSS, and the preinitiation complex is formed. By contrast, transcription regulation is bacterial-like, with over half of the identified archaeal transcription factors having at least one bacterial homolog.

### Methanogenic Archaea and Methanogenesis

The prokaryotic host cell provided herein can optionally be a methanogenic archaeal cell. Methanogenic archaea are also known as methanogens. Methanogenic archaea can produce methane (in a process known as methanogenesis) from CO₂, using H₂ as the reducing agent. Methanogens that use carbon dioxide as a source of carbon, and hydrogen as a reducing agent are known as hydrogenotrophic methanogens. Alternatives to CO₂ include, but are not limited to: acetate; formate; methanol; and methylamines. Methanogens play an important role in the ecosystems of anaerobic environments, by removing the products of other anaerobes, including excess hydrogen. The thermal breakdown of water and water radiolysis are other possible sources of hydrogen. Methanogens may thrive in environments in which electron acceptors other than CO₂ (for example, oxygen, Fe(III) ions, nitrate ions, and sulfate ions) are depleted.

The overall reduction of carbon dioxide to methane in the presence of hydrogen (H₂/CO₂ methanogenesis) can be expressed as:

CO₂+4H₂→CH₄+2 H₂O

In the earliest stage of H₂/CO₂ methanogenesis, CO₂ binds to methanofuran (MF) and is reduced to formyl-MF with oxidation of reduced ferredoxin (Fd_{red}²⁻) to oxidized ferredoxin (Fdₒₓ). The reaction is catalyzed by formyl-MF dehydrogenase (Fmd):

CO₂+Fd_{red}²⁻+MF+2H⁺→HCO-MF+Fdₒₓ+H₂O

Subsequently, the formyl moiety from formyl-MF is transferred to the coenzyme tetrahydromethanopterin (H₄MPT) thereby forming formyl-H₄MPT. The reaction is catalyzed by formyl transferase (Ftr):

HCO-MF+H₄MPT→HCO—H₄MPT+MF

Formyl-H₄MPT is then dehydrated to methenyl-H₄MPT. This step is catalyzed by methenyl-H₄MPT cyclohydrolase (Mch):

HCO—H₄MPT+H⁺→CH—H₄MPT⁺+H₂O

Methenyl-H₄MPT is then converted to methylene-H₄MPT with oxidation of coenzyme F₄₂₀, assisted by F₄₂₀-dependent methenyl-H₄MPT cyclohydrolase (Mtd).

CH—H₄MPT⁺+F₄₂₀H₂→CH₂=H₄MPT+F₄₂₀+H⁺

Alternatively, the formation of methylene-H₄MPT may be assisted by F₄₂₀-independent methenyl-H₄MPT cyclohydrolase (Hmd).

CH—H₄MPT⁺+H₂→CH₂=H₄MPT+H⁺

Subsequently, methylene-H₄MPT is reduced to methyl-H₄MPT with oxidation of coenzyme F₄₂₀, which reaction is catalyzed by methylene-H₄MPT reductase (Mer):

CH₂═H₄MPT+F₄₂₀H₂→CH₃—H₄MPT+F₄₂₀

Following this, a methyl group is transferred from methyl-M₄MPT to coenzyme M. The reaction is catalyzed by methyl-H₄MPT:coenzyme M methyltransferase (Mtr):

CH₃—H₄MPT+HS-CoM→CH₃—S-CoM+H₄MPT

The final steps of H₂/CO₂ methanogenic are assisted by coenzymes N-7 mercaptoheptanoylthreonine phosphate (coenzyme B or HS-CoB) and coenzyme F₄₃₀. H₂ donates electrons to the mixed disulfide of HS-CoM and regenerates coenzyme M. The relevant enzymes are methyl-coenzyme M reductase (Mcr) and heterodisulfide reductase (Hdr) complex:

CH₃—S-CoM+HS-CoB→CH₄+CoM-S—S—CoB

CoM-S—S-CoB+Fdₒₓ+2H₂→CoM-SH+HS-CoB+Fd_{red}²⁻+2H⁺

It will be appreciated that methanogenesis from different starting materials than H₂/CO₂ can occur via analogous processes.

The methanogenic archael host cell can optionally be a cell of one of the following families: Methanomicrobiaceae; Methanospirillaceae; Methanocorpusculaceae; Methanoregulaceae; Methanocalculaceae; Methanosarcinaceae; Methanosaetaceae; Methermicoccaceae; Methanopyraceae; Methanocellaceae; and Methanomassiliicoccaceae.

The methanogenic archaeal host cell can optionally be a cell of one of the following genera: Methanobacterium; Methanobrevibacter; Methanococcus; Methanocorpusculum; Methanoculleus; Methanoflorens; Methanofollis; Methanogenium; Methanomicrobium; Methanopyrus; Methanoregula; Methanosaeta; Methanosarcina; Methanosphaera; Methanospirillium; Methanothermobacter; and Methanothrix.

The methanogenic archaeal host cell can optionally be a cell of: Methanobacterium bryantii; Methanobacterium formicum; Methanobrevibacter arboriphilicus; Methanobrevibacter gottschalkii; Methanobrevibacter ruminantium; Methanobrevibacter smithii; Methanocalculus chunghsingensis; Methanococcoides burtonii; Methanococcus aeolicus; Methanococcus voltae; Methanocaldococcus jannaschii; Methanococcus maripaludis; Methanococcus vannielii; Methanocorpusculum labreanum; Methanoculleus bourgensis (Methanogenium olentangyi & Methanogenium bourgense); Methanoculleus marisnigri; Methanoflorens stordalenmirensis; Methanofollis liminatans; Methanogenium cariaci; Methanogenium frigidum; Methanogenium organophilum; Methanogenium wolfei; Methanomicrobium mobile; Methanopyrus kandleri; Methanoregula boonei; Methanosaeta concilii; Methanosaeta thermophila; Methanosarcina acetivorans; Methanosarcina barkeri; Methanosarcina mazei; Methanosphaera stadtmanae; Methanospirillium hungatei; Methanothermobacter defluvii; Methanothermobacter thermautotrophicus; Methanothermobacter marburgensis; Methanothermobacter thermoflexus; Methanothermobacter wolfei; or Methanothrix soehngenii. The host cell can be a Methanococcus cell. The host cell can be a Methanococcus maripaludis cell.

### Cell Cultures

Provided herein is a cell culture, comprising prokaryotic host cells as provided herein.

The cell culture may be an anaerobic cell culture. For example, the cell culture may be under an atmosphere comprising, or consisting of, a mixture of N₂ and CO₂. N₂ and CO₂ may be present in a ratio by volume of about 70-90% N₂:about 10-30% CO₂; about 75-90% N₂:about 10-25% CO₂; or about 75-85% N₂:about 15-25% CO₂. For example, N₂ and CO₂ may be present in a ratio by volume of about 80% N₂:about 20% CO₂. Where the cell culture is under an atmosphere comprising N₂ and CO₂, the pressure of the atmosphere may be about 50-200 kPa, 50-150 kPa, or 70-130 kPa, for example about 103 kPa.

The cell culture, including its atmosphere, may optionally contain a limited supply of electron acceptors other than CO₂ (for example, oxygen, Fe(III) ions, nitrate ions, and sulfate ions). The cell culture, including its atmosphere, may be free or substantially free of electron acceptors other than CO₂ (for example, oxygen, Fe(III) ions, nitrate ions, and sulfate ions). For example, the medium of the cell culture may be sparged with N₂ and/or CO₂ prior to inoculation, thereby removing all or substantially all O₂.

The cell culture may optionally comprise a medium that contains a source of carbon for reduction, including, but not limited to: CO₂; acetate; formate; methanol; and methylamines, which may be introduced to the medium by any appropriate means. The cell culture may optionally comprise a medium that contains a source of reductant, including, but not limited to, H₂ or cysteine chloride.

The cell culture may optionally comprise a medium comprising (e.g., having an atmosphere comprising) H₂ and CO₂ (in this case, the medium may conveniently be termed an ‘H₂/CO₂ medium’). Where the cell culture is supplied with H₂ and CO₂, these may be present in a ratio by volume of about 70-90% H₂:about 10-30% CO₂; about 75-90% H₂:about 10-25% CO₂; or about 75-85% H₂:about 15-25% CO₂. For example, H₂ and CO₂ may be present in a ratio by volume of about 80% H₂:about 20% CO₂. One or both of H₂ and CO₂ may optionally be supplied to the culture medium intermittently. For example, prior to inoculation, the atmosphere of the culture medium may be pressurized with H₂ and/or CO₂. Subsequent to inoculation, refills of H₂ and/or CO₂ can be provided at suitable time intervals, for example at 12 hour, 24 hour, or 36 hour time intervals. Where both CO₂ and H₂ are supplied intermittently, CO₂ may be supplied at different time intervals to H₂. One or both of H₂ and CO₂ may be supplied to the culture medium continuously. Thus, both CO₂ and H₂ may be supplied to the culture medium intermittently; or CO₂ may be supplied intermittently while H₂ is supplied continuously; or H₂ may be supplied intermittently while CO₂ is supplied continuously; or both CO₂ and H₂ may be supplied continuously. It will be appreciated that where a supply of CO₂ and/or H₂ is continuous, it need not be never-ending, and could be paused, for example during start-up or shut-down of the cell culture (e.g., for the purpose of maintenance of the apparatus comprising the cell culture). Where the cell culture is under an atmosphere comprising H₂ and CO₂, the pressure of the atmosphere may be about 250-300 kPa, 260-290 kPa, or 270-280 kPa, for example about 276 kPa.

The cell culture may optionally be conveniently located close to an industrial source of CO₂, for example a power plant (e.g., a fossil fuel or a biomass power plant) or petroleum refinery. Additionally or alternatively, CO₂ for provision to the cell culture may be captured from the atmosphere. CO₂ capture may be carried out by any appropriate means known to one of ordinary skill in the art, including but not limited to membrane capture; oxyfuel combustion; absorption; multiphase absorption; adsorption; chemical looping combustion; calcium looping; and cryogenic capture.

The cell culture may optionally comprise a medium comprising formate (which may conveniently be termed a ‘formate medium’). For example, the medium may comprise sodium formate. The medium may comprise about 0.01-1 M, 0.01-0.8 M, 0.01-0.6 M, 0.1-0.6 M, or 0.2-0.6 M formate. For example, the medium may comprise about 0.4 M formate, such as sodium formate. In addition to formate, the medium may comprise a reductant, for example cysteine hydrochloride, coenzyme M, or dithiothreitol. The medium may comprise about 1-10 mM, 1-5 mM, or 2-5 mM of a reductant. For example, the medium may comprise about 3 mM of a reductant, such as cysteine hydrochloride. Where the medium comprises formate, it may be under an atmosphere comprising, or consisting of, a mixture of N₂ and CO₂, as described hereinabove.

The cell culture medium may optionally comprise a buffer, for example one or more of Tris/HCl (wherein Tris is 2-amino-2-hydroxy-methyl-propane-1,3-diol), glycine/NaOH, and glycylglycine/NaOH.

The cell culture medium may optionally comprise a sulfur source, such as a sulfide, for example sodium sulfide. The medium may optionally comprise 1-10 mM, 1-5 mM, or 2-5 mM sulfide. For example, the medium may comprise about 3 mM sulfide, such as sodium sulfide.

The cell culture medium may optionally comprise phosphates, for example inorganic phosphates. The concentration of phosphates in the medium may optionally be kept constant over time. Alternatively, the concentration of phosphates in the medium may vary over time. For example, the concentration of phosphates in the medium may be adjusted to an initial value prior to inoculation, and decline following inoculation as phosphate is taken up by the cells. The initial concentration of phosphate may be less than 500 μM, less than 400 μM, less than 300 μM, or less than 200 μM. The supply of phosphates may decline over time as phosphate is taken up by cells, but be replenished intermittently, for example at intervals of about 36 hours, 48 hours, or 72 hours.

The cell culture may be kept at a temperature of from about 0-100° C., 10-60° C., 20-50° C., 30-50° C., or 35-45° C., for example at about 37° C.

The cell culture may be a co-culture, comprising one or more further microbial cells in addition to the prokaryotic host cells provided herein. The additional microbial cells may be bacterial cells. The additional microbial cells may be eukaryotic cells. The additional microbial cells may be prokaryotic cells. Thus, the additional microbial cells may be of the same or a different domain to the prokaryotic host cells provided herein. The additional microbial cells may be archaeal cells, for example methanogenic archaeal cells. For example, both the host cells provided herein and the additional microbial cells may be methanogenic archaeal cells, in which case they may be cells of the same strain (e.g., Methanococcus maripaludis) or of different strains.

The cell culture may optionally be provided in a culture tube having a volume of at least about 1 mL, 2 mL. 5 mL, 10 mL, 25 mL, 50 mL or 100 mL and up to about 1 L, 500 mL, 200 mL, 100 mL, 50 mL or 30 mL. For example, the tube may have a volume of about 28 mL. Suitable tubes will be familiar to one of ordinary skill in the art. The tube may be acid-washed in 1% (v/v) hydrochloric acid (HCl) overnight before use. A rubber stopper and aluminum crimp seal may be used to seal the tube.

The cell culture may be of an industrial scale. For example, the cell culture may occupy a container having a volume of up to about 1 m³, 2 m³, 4 m³, 5 m³, 6 m³, 7 m³, 8 m³, 9 m³, 10 m³, 20 m³, 30 m³, 40 m³, 50 m³, 60 m³, 70 m³, 80 m³, 90 m³, 100 m³, 200 m³, 300 m³, 400 m³, 500 m³, 600 m³, 700 m³, 800 m³, 900 m³, or 1000 m³. For example, the cell culture may occupy a container having a volume of at least about 0.1 m³, 0.2 m³, 0.5 m³, 1 m³, 2 m³, 4 m³, 5 m³, 6 m³, 7 m³, 8 m³, 9 m³ or 10 m³.

### Methods for Inducing Expression of an Exogenous Gene in a Prokaryotic Cell Culture

Provided herein is a method for inducing expression of an exogenous gene in a prokaryotic cell culture provided herein. The method comprises lowering the concentration of phosphate, for example inorganic phosphate (e.g. K₂HPO₄) in the culture, from an initial phosphate concentration.

The initial phosphate concentration may optionally be selected depending on the environmental conditions under which the cells exist. For example, the initial phosphate concentration may be selected depending on the type of medium in which the cells are cultured. It will be appreciated that the initial phosphate concentration selected when the medium comprises formate (a “formate medium”) may be the same as, or may differ from, the initial phosphate concentration selected when the medium does not comprise formate but is under an atmosphere of H₂ and CO₂ (an “H₂/CO₂ medium”).

The initial phosphate concentration may optionally be less than about 1 mM, 750 μM, 500 μM, 400 μM, 300 μM, 290 μM, 280 μM, 270 μM, 260 μM, 250 μM, 240 μM, 230 μM, 220 μM, 200 μM, 190 μM, 180 μM, 170 μM, 160 μM, 150 μM, 140 μM, 130 μM, 120 μM, 110 μM, 100 μM, 90 μM, 80 μM, 70 μM, 60 μM, 50 μM, 40 μM, 30 μM, 20 μM, or 10 μM. The initial phosphate concentration may optionally be in a range of about 50-750 μM, 50-500 μM, 50-300 μM, 50-200 μM, or 80-150 μM.

By way of example, when the medium comprises formate, the initial phosphate concentration may be less than about 300 μM, 290 μM, 280 μM, 270 μM, 260 μM, 250 μM, 240 μM, 230 μM, 220 μM, 200 μM, 190 μM, 180 μM, 170 μM, 160 μM, 150 μM, 140 μM, 130 μM, 120 μM, 110 μM, 100 μM, 90 μM or 80 μM. By way of example, when the medium comprises (e.g., the atmosphere of the medium comprises) H₂ and CO₂, the initial phosphate concentration may be less than about 200 μM, 190 μM, 180 μM, 170 μM, 160 μM, 150 μM, 140 μM, 130 μM, 120 μM, 110 μM, 100 μM, 90 μM or 80 μM.

In some instances, a promoter provided herein can mediate transcription of an operably linked nucleic acid sequence in a prokaryotic cell, during culturing of the host cell under conditions of phosphate (e.g., inorganic phosphate) depletion, but not during culturing of the cell under phosphate replete conditions. For example, a promoter provided herein can mediate transcription of an operably linked nucleic acid sequence in a methanogenic archaeal cell (e.g., a Methanococcus maripaludis cell) cultured under conditions of inorganic phosphate depletion, but not under inorganic phosphate replete conditions.

In this way, when the concentration of phosphate (e.g., inorganic phosphate) is high, the host cells grow; at this stage, recombinant gene expression is low. When the concentration of phosphate drops to limiting concentration during growth, recombinant gene expression can be upregulated at the same time as biomass production is limited. Thus, growth is decoupled from expression, allowing the production of proteins or metabolites inhibitory to growth.

It will be appreciated that, apart from manipulating the concentrations of phosphate and other growth conditions, protein expression can optionally be controlled by modifying the transcription initiation rate. It will similarly be appreciated that protein expression can optionally be controlled by modifying the translation initiation rate. For example, protein expression can be controlled by altering one or more of: the core promoter elements, 5′ untranslated region (UTR), ribosome binding sites (RBS), and start codon.

Thus, provided herein is a practical regulatory system for protein overexpression. The present system is particularly suitable for large scale (e.g. industrial) cultures and thus for large-scale protein and chemical production.

### Transformation Methods

The present invention also provides transformation methods in which a prokaryotic cell (in particular one of the cells listed above under the heading “cells”) is transformed with an expression vector provided herein. A method provided herein comprises introducing a vector provided herein into a prokaryotic cell and selecting for a transformed prokaryotic cell. A method is also provided herein for co-transforming a prokaryotic cell, comprising introducing the expression cassette provided herein and a nucleic acid sequence encoding a selectable marker into a prokaryotic cell, and selecting for the presence of the selectable marker in a transformed prokaryotic cell, thereby providing a prokaryotic cell transformed with the expression cassette.

The expression vector may be introduced by methods familiar to those skilled in the art. Relevant processes include, as non-limiting examples: natural transformation; polyethylene glycol (PEG) mediated transformation; E. coli conjugation; liposome-mediated transformation; PEG spheroplasting; electroporation; CaCl₂ mediated transformation; and heat shock. Thus, by way of example, host cells provided herein (for example, methanogenic archaeal cells, such as Methanococcus cells, e.g., Methanococcus maripaludis cells) may be transformed by a method such as E. coli conjugation.

Selectable markers contemplated herein include any gene that confers a phenotype on a cell in which it is expressed to facilitate the selection of cells that are transfected or transformed with a nucleic acid construct of the invention. The term may also be used to refer to gene products that effectuate said phenotypes. Examples of selectable markers include genes conferring resistance to antibiotics or antimicrobials (e.g., puromycin, neomycin, 8-azahypoxanthine, 6-azauracil). Further examples of selectable markers include genes that may be used in auxotrophic strains (e.g., histidine auxotrophy) or to confer other metabolic effects.

### Additional Embodiments

Embodiment 1. An isolated DNA molecule, comprising a sequence having at least about 80% identity to at least about 80 contiguous nucleotides of SEQ ID No. 1.
Embodiment 2. An isolated DNA molecule comprising a sequence having at least about 80% identity to at least about 200 contiguous nucleotides of SEQ ID No. 2.
Embodiment 3. An expression cassette comprising: a promoter comprising the DNA molecule according to Embodiment 1 or Embodiment 2; and a gene encoding a polypeptide or a functional RNA sequence, wherein the gene is operably linked to the promoter.
Embodiment 4. The expression cassette according to Embodiment 3, wherein the gene encodes an enzyme associated with methanogenesis or reverse methanogenesis.
Embodiment 5. A vector comprising an expression cassette according to Embodiment 3 or Embodiment 4.
Embodiment 6. The vector according to Embodiment 5, further comprising one or more selectable markers and/or reporter genes.
Embodiment 7. A prokaryotic host cell comprising the expression cassette according to Embodiment 3 or Embodiment 4.
Embodiment 8. The prokaryotic host cell according to Embodiment 7, wherein the cell is an archaeal cell.
Embodiment 9. The prokaryotic host cell according to Embodiment 8, wherein the cell is a methanogenic archaeal cell.
Embodiment 10. The prokaryotic host cell according to Embodiment 9, wherein the cell is a Methanococcus cell.
Embodiment 11. The prokaryotic host cell according to Embodiment 9, wherein the cell is a cell of: Methanobacterium bryantii; Methanobacterium formicum; Methanobrevibacter arboriphilicus; Methanobrevibacter gottschalkii; Methanobrevibacter ruminantium; Methanobrevibacter smithii; Methanocalculus chunghsingensis; Methanococcoides burtonii; Methanococcus aeolicus; Methanococcus voltae; Methanocaldococcus jannaschii; Methanococcus maripaludis; Methanococcus vannielii; Methanocorpusculum labreanum; Methanoculleus bourgensis (Methanogenium olentangyi & Methanogenium bourgense); Methanoculleus marisnigri; Methanoflorens stordalenmirensis; Methanofollis liminatans; Methanogenium cariaci; Methanogenium frigidum; Methanogenium organophilum; Methanogenium wolfei; Methanomicrobium mobile; Methanopyrus kandleri; Methanoregula boonei; Methanosaeta concilii; Methanosaeta thermophila; Methanosarcina acetivorans; Methanosarcina barkeri; Methanosarcina mazei; Methanosphaera stadtmanae; Methanospirillium hungatei; Methanothermobacter defluvii; Methanothermobacter thermautotrophicus; Methanothermobacter marburgensis; Methanothermobacter thermoflexus; Methanothermobacter wolfei; or Methanothrix soehngenii.
Embodiment 12. The prokaryotic host cell according to Embodiment 10, wherein the cell is a Methanococcus maripaludis cell.
Embodiment 13. A cell culture comprising prokaryotic host cells according to any one of Embodiments 7 to 12.
Embodiment 14. A method for inducing expression of an exogenous gene in a prokaryotic cell culture according to Embodiment 13, the method comprising lowering the concentration of inorganic phosphates in the culture from an initial concentration.
Embodiment 15. The method according to Embodiment 14, wherein the concentration of inorganic phosphate is lowered from an initial concentration in a range of from about 10 to 300 μM.
Embodiment 16. The method according to Embodiment 15, wherein the concentration of inorganic phosphate is lowered from an initial concentration in a range of from about 80 to 150 μM.
Embodiment 17. A method for transforming a prokaryotic cell, the method comprising: introducing a vector according to Embodiment 5 or Embodiment 6 into the prokaryotic cell; and selecting for a transformed prokaryotic cell.
Embodiment 18. A method for co-transforming a prokaryotic cell, the method comprising: introducing an expression cassette according to Embodiment 3 or Embodiment 4 and a nucleic acid sequence encoding a selectable marker into the prokaryotic cell; and selecting for the presence of the selectable marker in a transformed prokaryotic cell to provide a prokaryotic cell transformed with the expression cassette.

### EXAMPLES

The following examples are merely illustrative, and do not limit this disclosure in any way.

### Methods.

### Microbial Strains and Culture Conditions.

Methanococcus maripaludis strains (Table 1) were grown anaerobically at 37° C. in an atmosphere of 80% N₂: 20% CO₂ in minimal formate or H₂/CO₂ medium as described in Long, F. et al., A Flexible System for Cultivation of Methanococcus and Other Formate-Utilizing Methanogens, Archaea 2017, 7046026, and Sarmiento, F. et al., Genetic systems for hydrogenotrophic methanogens, Methods Enzymol 494, 43-73, the contents of which are incorporated herein by reference in their entirety. For low inorganic phosphate (LPᵢ) treatment, the concentration of potassium phosphate dibasic (K₂HPO₄) in the medium was reduced from 800 μM to 40 μM or 80 μM, while the high Pᵢ treatment (HPᵢ) remained at 800 μM. 28 mL culture tubes or 160 mL culture bottles with a 28 mL tube side-arm were acid-washed in 1% (v/v) hydrochloric acid (HCl) overnight before use. Rubber stoppers and aluminum crimp seals were used to seal the tubes and bottles. The plasmids (Table 1) were maintained in the recombinant M. maripaludis strains by adding 2.5 μg/mL puromycin to the medium unless otherwise stated. The strains were pre-grown in 5 mL low Pᵢ medium except otherwise stated. After 12-24 hours, 4% inoculum was transferred into the low or high Pᵢ medium. All cultures were prepared at least in triplicate. Growth was monitored via optical density at 600 nm (OD₆₀₀) with a spectrophotometer.

TABLE 1
Microbial strains and plasmids.

| Name | Description |
|---|---|
| pMEV4m | mCherry reporter vector for the M. maripaludis S0001 |
| pMEV5mT | Postfix of Tsl terminator after mCherry gene in pMEV4m¹ |
| pMEV5mT-P243 | Replaced PhmvA in pMEV4m with wild type full-length |
| | 243 bp Ppst |
| pMEV5mT-P93 | Replaced PhmvA in pMEV4m with 93 bp Ppst |
| pMEV5mT-P85 | Replaced PhmvA in pMEV4m with 85 bp Ppst |
| pMEV5mT-P80 | Replaced PhmvA in pMEV4m with 80 bp Ppst |
| pMEV5mT-P73 | Replaced PhmvA in pMEV4m with 73 bp Ppst |
| pMEV5mT-P67 | Replaced PhmvA in pMEV4m with 67 bp Ppst |
| pMEV5mT-P93-BRE1 | Replaced pMEV5mT-P93 BRE with Pslp BRE |
| pMEV5mT-P93-BRE2 | Replaced pMEV5mT-P93 BRE with Pmmp1338 BRE |
| pMEV5mT-P93-BRE3 | Replaced pMEV5mT-P93 BRE with Pmmp0466 BRE |
| pMEV5mT-P93-UTR1 | Replaced pMEV5mT-P93 5′ UTR with slp 5′ UTR |
| pMEV5mT-P93-UTR2 | Replaced pMEV5mT-P93 5′ UTR with hisA 5′ UTR |
| pMEV5mT-P93-UTR3 | Replaced pMEV5mT-P93 5′ UTR with mcrB 5′ UTR |
| pMEV5mT-P93-UTR4 | Replaced pMEV5mT-P93 5′ UTR with MMP1338 5′ UTR |
| pMEV5mT-P93-UTR5 | Replaced pMEV5mT-P93 5′ UTR with MMP0466 5′ UTR |
| pMEV5mT-P93-5S | Delete the 6th base from mCherry start codon in |
| | pMEV5mT-P93 |
| pMEV5mT-P93-4S | Delete the 5th & 6th base from mCherry start codon in |
| | pMEV5mT-P93 |
| pMEV5mT-P93-TTG | Replaced mCherry ATG start codon in pPDS1 with TTG |
| pMEV5mT-P93-GTG | Replaced mCherry ATG start codon in pPDS1 with GTG |
| pMEV5mT-P243-TAP- | Replaced mCherry in pMEV5mT-P243 with TAP-tagged |
| mcrBCDGA | M. maripaludis mcr operon |
| pMEV5mT-P243-N- | Replaced mCherry in pMEV5mT-P243 with N-terminal |
| TAP-mmpX | TAP-tagged M. maripaludis MMP10 |
| pMEV5mT-P243-C- | Replaced mCherry in pMEV5mT-P243 with C-terminal |
| TAP-mmpX | TAP-tagged M. maripaludis MMP10 |
| S0001 | Expression host containing ORF1 from pURB500 |
| | integrated into the M. maripaludis S2 genome |
| 243 bp Ppst | Recombinant M. maripaludis S0001 strain hosting |
| | pMEV5mT-P243 |
| 93 bp Ppst | Recombinant M. maripaludis S0001 strain hosting |
| | pMEV5mT-P93 |
| 85 bp Ppst | Recombinant M. maripaludis S0001 strain hosting |
| | pMEV5mT-P85 |
| 80 bp Ppst | Recombinant M. maripaludis S0001 strain hosting |
| | pMEV5mT-P80 |
| 73 bp Ppst | Recombinant M. maripaludis S0001 strain hosting |
| | pMEV5mT-P73 |
| 67 bp Ppst | Recombinant M. maripaludis S0001 strain hosting |
| | pMEV5mT-P67 |
| BRE-slp | Recombinant M. maripaludis S0001 strain hosting |
| | pMEV5mT-P93-BRE1 |
| BRE-1338 | Recombinant M. maripaludis S0001 strain hosting |
| | pMEV5mT-P93-BRE2 |
| BRE-0466 | Recombinant M. maripaludis S0001 strain hosting |
| | pMEV5mT-P93-BRE3 |
| UTR-slp | Recombinant M. maripaludis S0001 strain hosting |
| | pMEV5mT-P93-UTR1 |
| UTR-his | Recombinant M. maripaludis S0001 strain hosting |
| | pMEV5mT-P93-UTR2 |
| UTR-mcr | Recombinant M. maripaludis S0001 strain hosting |
| | pMEV5mT-P93-UTR3 |
| UTR-1338 | Recombinant M. maripaludis S0001 strain hosting |
| | pMEV5mT-P93-UTR4 |
| UTR-0466 | Recombinant M. maripaludis S0001 strain hosting |
| | pMEV5mT-P93-UTR5 |
| UTR-pst-5S | Recombinant M. maripaludis S0001 strain hosting |
| | pMEV5mT-P93-5S |
| UTR-pst-4S | Recombinant M. maripaludis S0001 strain hosting |
| | pMEV5mT-P93-4S |
| TTG | Recombinant M. maripaludis S0001 strain hosting |
| | pMEV5mT-P93-TTG |
| GTG | Recombinant M. maripaludis S0001 strain hosting |
| | pMEV5mT-P93-GTG |
| P243-TAP-mcrBCDGA | Recombinant M. maripaludis S0001 strain hosting the |
| | pMEV5mT-P243-TAP-mcrBCDGA |
| P243-N-TAP-mmpX | Recombinant M. maripaludis ΔmmpX deletion strain |
| | hosting the pMEV5mT-P243-C-TAP-MMP10 |
| P243-N-TAP-mmpX | Recombinant M. maripaludis ΔmmpX deletion strain |
| | hosting the pMEV5mT-P243-N-TAP-MMP10 |

### Plasmid and Recombinant Strain Construction.

The plasmids and PCR primers used are listed in Table 1 and Table 2, respectively. All PCR amplifications were done either by Phusion® High-Fidelity DNA Polymerase (NEB, M0530) or Q5® High-Fidelity DNA Polymerase (NEB, M0515). All ligations were done with T4-ligase (NEB, M0202S). Plasmids pMEV5mT was made by postfixing the Tsl terminator (Table 1) after the mCherry reporter gene in the pMEV4m plasmid, where expression of mCherry was driven by the constitutive promoter PhmvA. The terminator was introduced by amplifying pMEV4m with the AflII-containing primers 0 mT-F/R, digested and ligated at AflII. The addition of the terminator was expected to separate the transcriptions of gene inserts and puromycin resistance pac cassette, which would minimize any pleiotropic effects especially in the event of strong and constitutive gene expression.

To develop a phosphate-dependent regulatory expression system, the wild-type pst promoter (Ppst; including the 5′-UTR for MMP1095) was cloned into the pMEV5mT by replacing the constitutive PhmvA promoter therein. First, a PhmvA and RBS region-less backbone was made by amplifying the pMEV5mT with primers 5p-F346/R46 where the restriction sites 5′-NdeI and 3′-HindIII were included. Then, the Ppst was amplified from the M. maripaludis genomic DNA with primers 5i-F4/R346 where restriction sites 5′-HindIII and 3′-NdeI were introduced. Lastly, the amplified products were restricted at HindIII and NdeI before cloning into the backbone restricted at the same sites. The resulting plasmid, pMEV5mT-P243 carried the wild-type Ppst. Two plasmids were made to further truncate the wild-type Ppst. This truncation was done by amplifying the pMEV5mT-P243 plasmid with primers P1-F/R and P2-F/P1-R where an XbaI site was included at both the 5′- and 3′-ends. The amplified products were then restricted by XbaI and ligated, which resulted in plasmids pMEV5mT-P93 and pMEV5mT-P67 carrying the 93 bp_Ppst and 67 bp_Ppst promoters, respectively. Plasmids pMEV5mT-P85, pMEV5mT-P80, pMEV5mT-P73, pMEV5mT-P93-BRE1-3 and pMEV5mT-P93-UTR1-5 were all synthesized by GenScript. These plasmids were cloned into E. coli Top10. The plasmids pMEV5mT-P93-5S and pMEV5mT-P93-4S were constructed by deleting the 6th nucleotide and 5th and 6th nucleotide from the start codon by amplifying the pMEV5mT-P93 with the primers SL1-F/R and SL2-F/SL1-R, respectively, using the Q5© Site-Directed Mutagenesis kit (E0554S). The plasmids pMEV5mT-P93-TTG and pMEV5mT-P93-GTG were constructed by amplifying the pMEV5mT-P93 with primers SC1-F/R and SC2-F/SC1-R, respectively, using the same kit. The primers were designed on the NEBaseChanger™ (http://nebasechanger.neb.com/) and are listed in Table 2. These plasmids were cloned into NEB® 5-alpha Competent E. coli using the NEBuilder® HiFi DNA Assembly Cloning Kit (NEB #E5520). The plasmids were purified from E. coli and transformed into M. maripaludis S0001. Colonies were picked from formate medium plates containing puromycin (see Long, F. et al., A Flexible System for Cultivation of Methanococcus and Other Formate-Utilizing Methanogens, Archaea 2017, 7046026).

TABLE 2
PCR primers.

| | | SEQ ID |
|---|---|---|
| Primers | Sequence (5′-3′) | No.: |
| 0mT-F | ATAAAAAACGCCCTATTCGTAATATACTATTC | SEQ ID |
| | | No. 7 |
| 0mT-R | CTGCAGCGGCCGCTACTAGTT | SEQ ID |
| | | No. 8 |
| 5p- | ATGGTTTCAAAAGGAGAAGAAGATA | SEQ ID |
| F346 | | No. 9 |
| 5p-R46 | CTAGAACCGGGCGCGAATTC | SEQ ID |
| | | No. 10 |
| 5i-F4 | GGGAAAAAGCTTGAATCCTCCATTCGTTCAAT | SEQ ID |
| | TC | No. 11 |
| 5i- | ATGAGACACCTCCCAGGTTTAT | SEQ ID |
| R346 | | No. 12 |
| P1-F | GGGAAATCTAGATTTATTCATAAAATAGGAAC | SEQ ID |
| | TAAAATACCAAAATAGAAAC | No. 13 |
| P2-F | GGGAAATCTAGACAAAATAGAAACATTTATAT | SEQ ID |
| | ACATTTGATGTGTAGGG | No. 14 |
| P1-R | GGGAAAGTCTAGAACCGGGCGCGAATT | SEQ ID |
| | | No. 15 |
| SL1-F | CTCATATGGTTTCAAAAGGAG | SEQ ID |
| | | No. 16 |
| SL2-F | TCATATGGTTTCAAAAGGAGAAG | SEQ ID |
| | | No. 17 |
| SL1-R | CACCTCCCAGGTTTATGG | SEQ ID |
| | | No. 18 |
| SC1-F | GGTGTCTCATTTGGTTTCAAAAG | SEQ ID |
| | | No. 19 |
| SC2-F | GGTGTCTCATGTGGTTTCAAAAG | SEQ ID |
| | | No. 20 |
| SC1-R | TCCCAGGTTTATGGTATAC | SEQ ID |
| | | No. 21 |
| Vector- | ACTAGTAGCGGCCGCTGCAGGATATA | SEQ ID |
| F | | No. 22 |
| Vector- | ATGAGACACCTCCCAGGTTTATGGTATACC | SEQ ID |
| R | | No. 23 |
| BDC-F | AAACCTGGGAGGTGTCTCATATGGTAAAGTAT | SEQ ID |
| | GAAGATAAGATAAG | No. 24 |
| BDC-R | TAAGATCACCTTAACCTAAATGTTTTTTTATG | SEQ ID |
| | CTTTC | No. 25 |
| GA-F | AAAGTCAGGTATGGCATACACGCCTCAG | SEQ ID |
| | | No. 26 |
| GA-R | CTGCAGCGGCCGCTACTAGTTTATTTAGCAGG | SEQ ID |
| | TAAGATAACGTCTC | No. 27 |
| TAP- | GGTGATCTTATGGACTACAAGGACCACGACGG | SEQ ID |
| ATG-F | C | No. 28 |
| TAP- | ACCTGACTTTTCGAACTGGGGGTGGCT | SEQ ID |
| ATG-R | | No. 29 |
| TAP-F | TTTAGGTTAAGGTGATCTTATGGACTAC | SEQ ID |
| | | No. 30 |
| TAP-R | TGTATGCCATACCTGACTTTTCGAACTG | SEQ ID |
| | | No. 31 |
| N-mmpX- | AAAGTCAGGTATGAACCAAAAGTTTGGATTTA | SEQ ID |
| F | ATAATATTG | No. 32 |
| N-mmpX- | CTGCAGCGGCCGCTACTAGTTTAAATAGGATT | SEQ ID |
| R | TCCAAAGAAGTTTATTTTTTC | No. 33 |
| N- | AAACCTGGGAGGTGTCTCATATGGACTACAAG | SEQ ID |
| TAP-F | GACCAC | No. 34 |
| N- | TTTGGTTCATACCTGACTTTTCGAACTG | SEQ ID |
| TAP-R | | No. 35 |
| C-mmpX- | AAACCTGGGAGGTGTCTCATATGAACCAAAAG | SEQ ID |
| F | TTTGGATTTAATAATATTG | No. 36 |
| C-mmpX- | CCTTGTAGTCAATAGGATTTCCAAAGAAGTTT | SEQ ID |
| R | ATTTTTTC | No. 37 |
| C- | AAATCCTATTGACTACAAGGACCACGAC | SEQ ID |
| TAP-F | | No. 38 |
| C- | CTGCAGCGGCCGCTACTAGTTTACTTTTCGAA | SEQ ID |
| TAP-R | CTGGGG | No. 39 |
| mCherry- | CCATACGAAGGTACACAGACAGCAA | SEQ ID |
| F | | No. 40 |
| mCherry- | TCTGCTGGGTGTTTAACGTATGC | SEQ ID |
| R | | No. 41 |

To put the mcrBDCGA under the phosphate-dependent expression system, the mCherry gene in pMEV5mT-P243 was replaced by the TAP-tagged mcrBDCGA. First, an mCherry-less backbone was amplified from pMEV5mT-P243 with the primers Vector-F/R while mcrBDC and mcrGA were amplified from M. maripaludis genome with the primers BDC-F/R and GA-F/R. All primers were designed using the NEBuilder assembly tool (https://nebuilder.neb.com/) with overlapping sequences on the 5′ end of both forward and reverse primers. The TAP tag (3×FLAG-Twin Strep Tag II) was first amplified from the plasmid AAVS1_Puro_PGK1_3×FLAG_Twin_Strep_EZH2 (Addgene, 79902) with the primers TAP-ATG-F/R which include ATG before the N-terminal 3×FLAG and the amino acids serine and glycine after the Twin strep tag IL. This product was amplified again with primers TAP-F/R including the overlapping sequences required for Gibson assembly. All PCR amplification was done using Q5® High-Fidelity DNA Polymerase (NEB #M0491) and digestion of the template DNA with DpnI (NEB #R0176). The PCR fragments were then assembled using the NEBuilder® HiFi DNA Assembly Master Mix (NEB #E2621) before cloning into NEB® 5-alpha Competent E. coli using the NEBuilder® HiFi DNA Assembly Cloning Kit (NEB #E5520). Similarly, the plasmids pMEV5mT-P243-N-TAP-MMPX and pMEV5mT-P243-C-TAP-MmpX were constructed by cloning the M. maripaludis mmpX into pMEV5mT-P243 with a N- or C-terminal TAP tag instead of the mCherry gene. The M. maripaludis mmpX was amplified from M. maripaludis genome using the primers N-mmp10-F/R or C-mmp10-F/R. The TAP tag was amplified from the plasmid AAVS1_Puro_PGK1_3×FLAG_Twin_Strep (Addgene, 68375) with the primers N-TAP-F/R or C-TAP-F/R. The two PCR products (mmpX gene and TAP tag) were assembled into mCherry-less backbone using the NEBuilder® HiFi DNA Assembly Cloning Kit (NEB #E5520), resulting in the expression plasmids pMEV5mT-P243-N-TAP-MMPX and pMEV5mT-P243-C-TAP-MMP10. All resulting plasmids except for mmpX plasmids purified from E. coli were transformed into M. maripaludis S0001, and colonies were picked from formate medium plates containing puromycin. The two recombinant mmpX plasmids were transformed into the ΔmmpX deletion strain of M. maripaludis, and colonies were also isolated on formate plates with puromycin (see Lyu, Z. et al., Posttranslational Methylation of Arginine in Methyl Coenzyme MReductase Has a Profound Impact on both Methanogenesis and Growth of Methanococcus maripaludis, J Bacteriol 202, 2020, which is incorporated herein by reference in its entirety). All the recombinant plasmids were verified by both PCR and sequencing.

mCherry Reporter Assay.

At each sampling point, 2 mL culture was anaerobically collected in a micro-centrifuge tube. The cells were harvested by centrifugation at 17,000 g for 1 minute. The cell pellet was resuspended in 200 μL of 25 mM PIPES (Piperazine-1,4-bis (2-ethanesulfonic acid) dipotassium salt) buffer adjusted to pH 6.8 using KOH. The cells were lysed by freezing in −20° C. and thawing at room temperature once before exposing the cell extract to oxygen overnight at 30° C. to allow for the mCherry chromophore to mature. The cell debris was separated from the cell extract by centrifugation at 17,000 g for 1 minute before collecting the cell extract containing the mCherry protein in the supernatant. Then, 100 μL of the supernatant was transferred into a Nunc® 96-well polystyrene, black, flat-bottom plate. Fluorescence reading was obtained using the BioTek Synergy™ Mx plate reader. Using the Gen 5 software, the excitation and emission wavelengths were set to 575 nm and 610 nm respectively with a bandwidth of 13.5 nm and gain of 75. The shake time was set to 10 seconds. The optics position was at the top, and the read height was 8 mm. The resulting fluorescent unit (FU) was normalized to the optical density at 600 nm (OD₆₀₀) using the formula: NFU=FU/OD₆₀₀. The FU of wild type M. maripaludis 50001 without the expression vector was used to correct for background noise.

RNA Extraction and cDNA Synthesis.

RNA was extracted using the Invitrogen PureLink RNA mini kit and the Invitrogen PureLink Pro 96 total RNA Purification kit, with column digestion of DNA using the Invitrogen PureLink DNase according to manufacturer instructions (Invitrogen, USA). Following the RNA purification, an additional DNase treatment was applied to all pure samples using Invitrogen TURBO DNase according to manufacturer's instructions, in order to completely remove any residual DNA. Total RNA was quantified using the Invitrogen Qubit RNA BR Assay kit. An Agilent 4150 TapeStation was used to confirm clear 16S and 23S rRNA peaks and evaluate RNA integrity before reverse transcription of the purified RNA was performed using the Applied Biosystems Power SYBR Green RNA-to-Ct 1-Step kit on the Applied Biosystems 7500 Fast Real-Time PCR system according to the manufacturers protocol (Applied Biosystems, USA). Data generated was analyzed with the 7500 Software v2.0.6 (Applied Biosystems, USA).

qRT-PCR.

The primers were created from the mCherry gene DNA sequence within the plasmid construct using ThermoFisher Primer Express software v3.0.1, with a melting temperature of 60° C., a primer length of 25 bp on the forward primer (mCherry-F) and 23 bp on the reverse primer (mCherry-R), and a GC content of 48%, and an amplicon product length of 125 bp with a 39% GC content. mCherry-F/R primers were evaluated in silica for similarity to the M. maripaludis genome (RefSeq NC_0035552) with BLASTn search, and evaluated for non-specific binding to the M. maripaludis genome with a qPCR assay using purified genomic gDNA of M. maripaludis. qPCR standard curves were created with 10-fold serial dilutions between 10⁹ and 10⁵ copies per reaction. qPCR DNA standards were made from pure mCherry plasmids. The plasmid DNA was quantified with the Invitrogen Qubit 1× dsDNA HS Assay kit. Triplicate qPCR reactions were performed for each control, standard, and sample. All assays had efficiencies between 90-99% and R² values above 0.99. Melt curves were analyzed for all reactions and showed no non-specific products. 40 cycle qPCR assays with no reverse transcriptase enzyme was run for all samples to check for amplification from contaminating DNA. No amplification was observed for all no template controls and all no RT enzyme control reactions. For qRT-PCR of mCherry, 0.5 ng of total RNA was used for each PCR reaction. All samples fit within the standard curve, and the amplification efficiency is 97% with R² of 0.99.

### Inorganic Phosphate Assay.

The Phosphate Colorimetric kit (Sigma-Aldrich MAK030-1KT) was modified to measure the Pᵢ concentration in the medium. This modification was required because salts of carboxylic acids, such as sodium formate and glycylglycine present in the medium, interfered with the assay. The interference was eliminated by the addition of HCl (final concentration of 0.9 M) to protonate the cations of these carboxylic acids. For generating the standard curve, 100 μL of phosphate-free medium was acidified with 100 μL of 1.8 M HCl before adding sufficient 0.5 mM K₂HPO₄ to generate 0, 5, 10, 20, 40 μM K₂HPO₄ standards. The mixture was performed in a Cellstar® tissue culture 96-well flat-bottom plate (Griener bio-one). To each well, 30 μL of the phosphate reagent was added and mixed. The reaction was incubated for 30 minutes at room temperature before measuring the absorbance at 650 nm using the BioTek Synergy™ Mx plate reader. For the assay of spent culture medium, after centrifugation to remove the cells, 100 μL of supernatant was acidified with 100 μL of 1.8 M HCl, 30 μL of the phosphate reagent was added, and the reaction was performed as described above. For the high Pᵢ cultures, the spent medium was diluted 10-fold with phosphate-free formate medium before acidification.

### Western Blot Analysis.

Cells expressing TAP-tagged recombinant MCR or MMPX proteins were grown in minimal formate medium with replete (800 μM) or limiting phosphate (40 or 80 μM). Cells were harvested by centrifugation at 17,000 g for 4 min and resuspended in 50 mM Tris-HCl [pH 7.6]. Cells were lysed by sonication for 10 cycles of 5 sec ON/OFF with the output set at 4 and the duty cycle set at 40%. The cell debris was removed by centrifugation at 17,000 g for 4 min, and the supernatant was collected. Protein concentrations were determined by a Pierce BCA protein assay kit (Thermo Fisher Scientific). Proteins were separated on precast 4-20% SDS-PAGE gels (Bio-Rad) and then transferred onto methanol-activated polyvinylidene difluoride (PVDF) membranes. Nonspecific binding was blocked with 5% milk in phosphate-buffered saline and 0.1% Tween 20 (PBST) for 1.5 h at room temperature. The PVDF membranes were then incubated with primary antibodies against the FLAG tag (1:2,000 dilution; catalog no. MA1-91878, Thermo Fisher Scientific) for 1.5 h at room temperature, washed three times for 15 min with PBST, and then incubated with horseradish peroxidase (HRP)-conjugated goat anti-mouse secondary antibodies (1:20,000 dilution; catalog no. 31430; Thermo Fisher Scientific) for 1 h. After additional washing with PBST, PVDF membranes were developed using the Western HRP substrate (ECL, catalog no. 32132; Thermo Fisher Scientific). The relative intensity of each immunoreactive band was estimated using ImageJ.

### Example 1: Tuning Recombinant Protein Production

### Bioinformatics Analysis of the Ppst

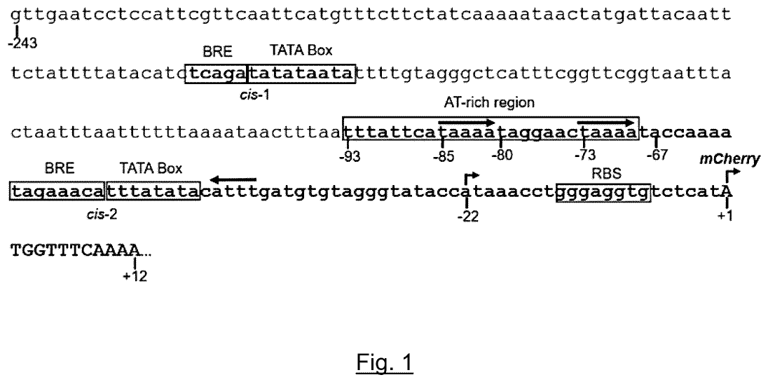

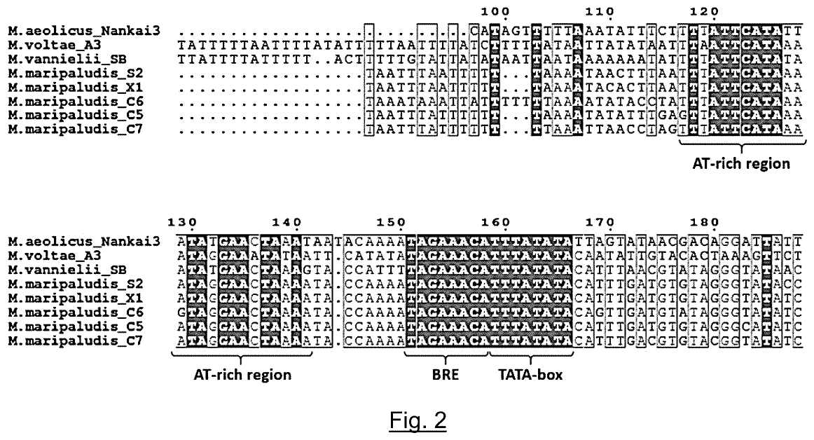

The genome of M. maripaludis contains a gene cluster consisting of five ORFs (MMP1095-MMP1099) predicted to encode the phosphate specific transport (Pst) system that is highly upregulated during phosphate limitation. The 243 bp intergenic sequence between MMP1094 and MMP1095 was presumed to contain the promoter or Ppst (FIG. 1). Bioinformatics analysis of this region in M. maripaludis and other closely related methanococci revealed conservation of the predicted cis-2 BRE and TATA box as well as an AT-rich region a few bases upstream of the BRE (FIG. 2). The TATA box was 23 bp upstream from the TSS. The operon also encoded a 22 bp 5′ UTR, which may play additional roles in phosphate-dependent regulation. The sequence analysis also identified both direct and inverted repeats within and immediately downstream of the AT-rich region as well as a potential second BRE and TATA box that may play a role in expression.

### Effect of the Conserved 25 bp AT-Rich Region on Phosphate-Dependent Regulation.

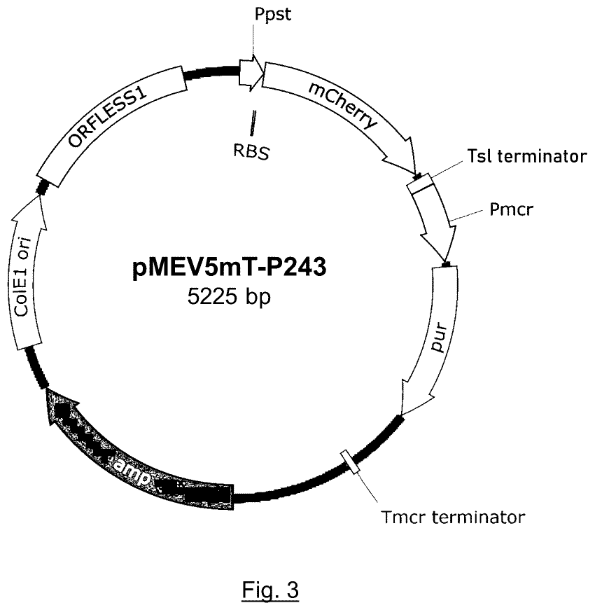

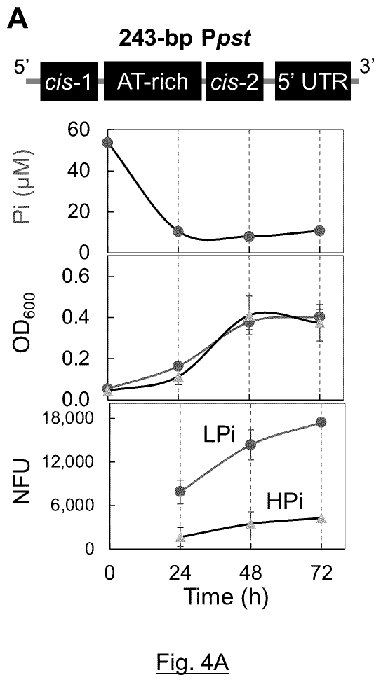

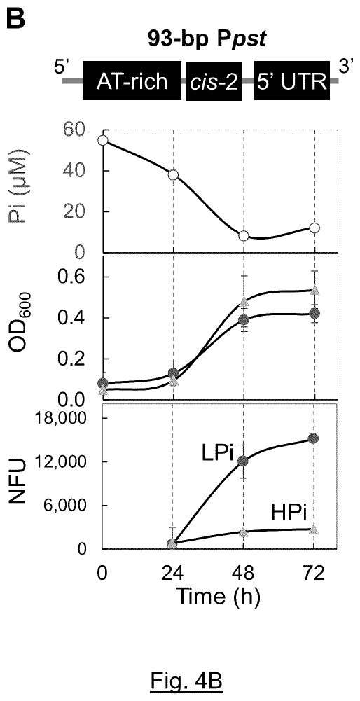

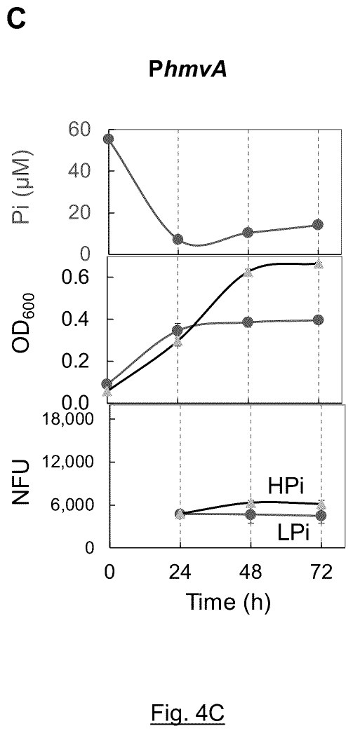

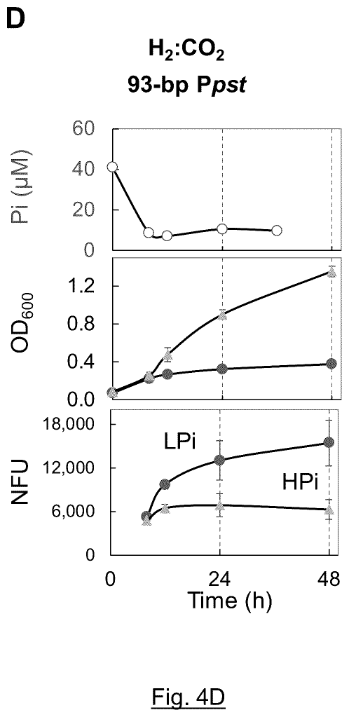

To test the functionality of the Ppst, the entire 243 bp intergenic region was cloned upstream of a mCherry reporter system optimized for M. maripaludis in the expression plasmid, pMEV5mT, resulting the pMEV5-Ppst-mCherry plasmid (FIG. 3). Then mCherry expression was investigated during growth with low (40 μM) and high (800 μM) initial Pᵢ concentrations (FIGS. 4A-D). It was expected that cultures grown with low initial Pᵢ concentrations would exhaust phosphate before the carbon and electron sources in the growth medium became limiting, resulting in upregulation of gene expressions controlled by Ppst. As expected, when the mCherry gene was under the control of the complete intergenic region, 243 bp Ppst, expression upon the depletion of free phosphate in the medium was 4-5-fold higher compared to growth under replete phosphate concentrations (FIG. 4A). By contrast, the strong constitutive promoter PhmvA showed no significant difference in mCherry expression between limiting and replete phosphate conditions (FIG. 4C). Of additional interest, after upregulation the expression from the 243 bp Ppst was >3-fold higher than from PhmvA, demonstrating that the 243 bp Ppst was a strong promoter. Moreover, growth and increases in expression continued even after the phosphate was depleted from the medium, suggesting that cells can accumulate and store phosphate intracellularly which can be used to support biomass biosynthesis.

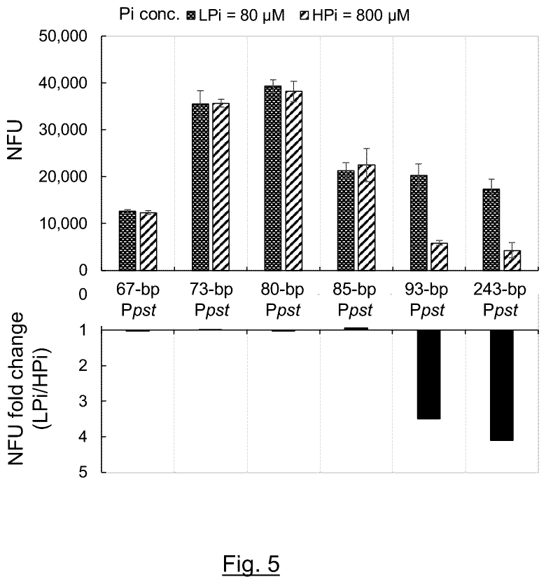

The predicted regulatory elements of Ppst were examined by a series of truncation mutants. To determine the role of the cis-1 BRE and TATA box, the 243 bp Ppst was truncated to create 93 bp Ppst. Expression from 93 bp Ppst was very similar to that from 243 bp Ppst, indicating that the cis-1 BRE and TATA site did not play a significant role in expression from the promoter (FIG. 4B). Further truncations were made to the AT-rich region at positions −85, −80 and −73. Although the truncated 80 bp Ppst and 73 bp Ppst elicited a 1.9-fold increase in mCherry expression compared to the full-length or 93 bp Ppst, expression was not significantly different under both limiting and replete phosphate conditions (FIG. 5). In fact, all four new truncations (85 bp Ppst, 80 bp Ppst, 73 bp Ppst, and 67 bp Ppst) lost the ability of phosphate-dependent regulation. Thus, the 93 bp intergenic sequence upstream of the start codon, consisting of the whole AT-rich region, cis-2 and 5′ UTR region, is the minimal regulatory promoter.

In formate-based medium, growth is limited by formate availability at an OD₆₀₀ of about 0.8. Thus, it was possible that expression might have been limited by the availability of an energy source late in growth. To test this possibility, expression was examined in H₂:CO₂-based medium where the electron donor was in great excess (FIG. 4E). Although the expression in the presence of high concentrations of phosphate was somewhat higher under these conditions, the increase in mCherry expression in both formate and H₂:CO₂ media late in growth was similar, implying that energy limitation did not affect expression in formate medium.

### Effect of Growth Conditions on Both Cell Yields and Expression Levels

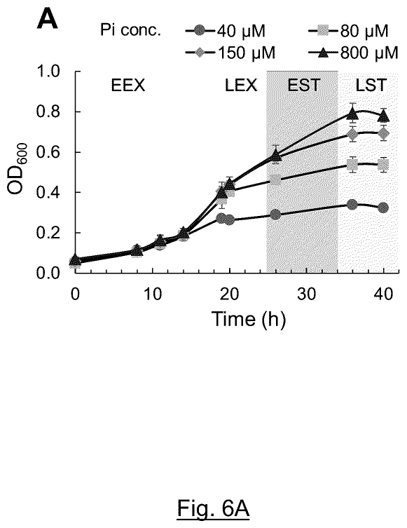

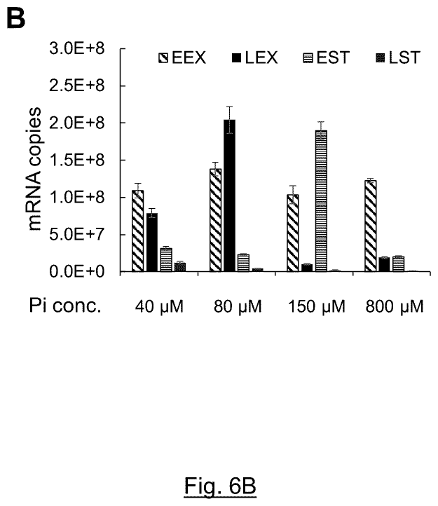

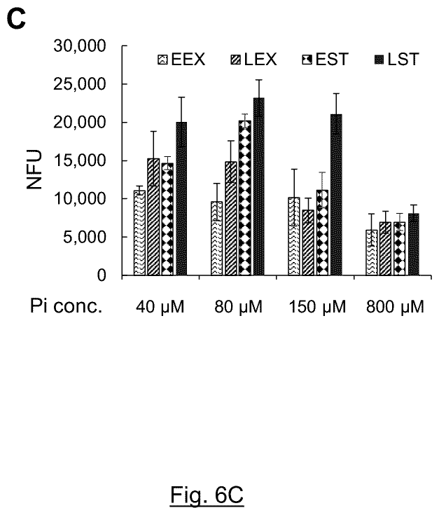

To optimize regulation from Ppst, the best growth conditions to achieve good cell yields and mCherry expression were investigated. Four initial phosphate concentrations (40, 80, 150, and 800 μM) were selected based on phosphate limitation studies in M. maripaludis^{34, 38, 39}. Increasing phosphate concentrations resulted in higher cell yields, with the highest optical density at 800 μM Pᵢ(OD₆₀₀, ˜0.8) and the lowest at 40 μM Pᵢ(OD₆₀₀, ˜0.3) (FIG. 6A). An initial concentration of 150 μM phosphate also slightly limited growth. Under all tested Pᵢ concentrations, the mRNA transcript levels and mCherry expression were similar during the early exponential (EEX) growth phase, suggesting that mCherry was initially formed in the low phosphate medium used to grow the inoculum (FIG. 6). At the lowest phosphate level of 40 μM, levels of mCherry transcripts slowly declined during growth while mCherry protein levels slowly increased (FIG. 6). By contrast, at the highest level of phosphate of 800 μM, the levels of transcripts rapidly decreased and the relative amount of mCherry protein remained constant. At the intermediate concentrations of 80 and 150 μM phosphate, large increases in the levels of mCherry transcripts during the LEX and EST growth phases, respectively, correlated with subsequent increases in mCherry protein levels. Thus, the highest recombinant protein production was achieved at initial Pᵢ concentrations between 80 and 150 μM at the late stationary (LST) phase.

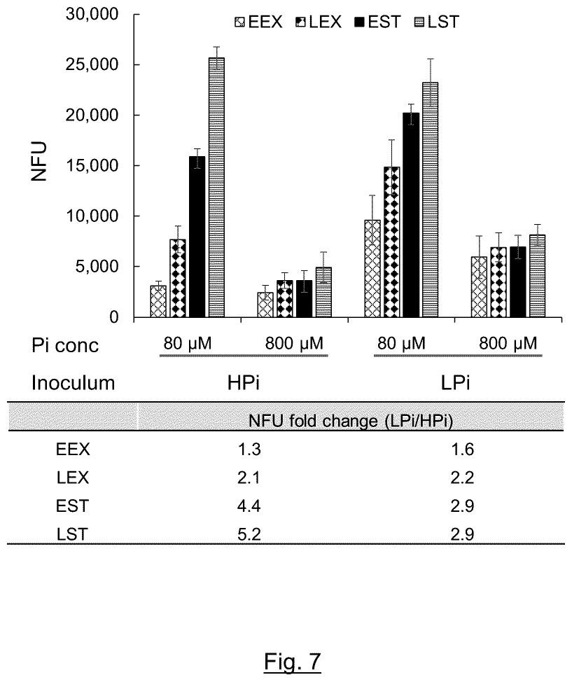

To further examine the role of phosphate in the inoculum, these experiments were repeated with inocula grown at high or low phosphate (FIG. 7). mCherry expression was 2-fold lower in the EEX growth phase when the inoculum was grown in high as compared to low phosphate medium, supporting the proposition that mCherry was formed prior to transfer in the previous experiment.

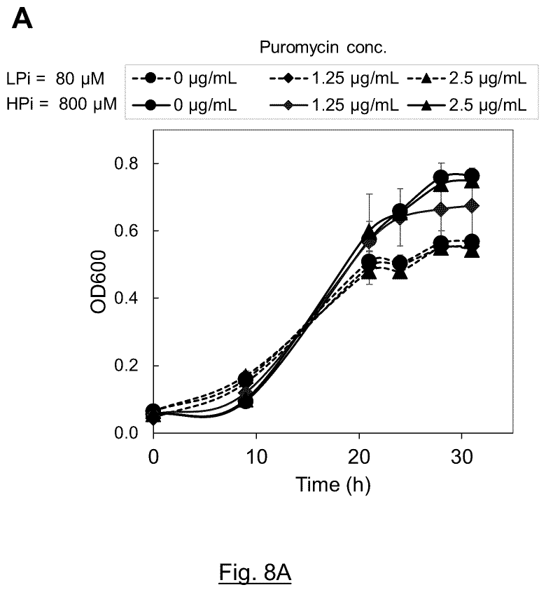

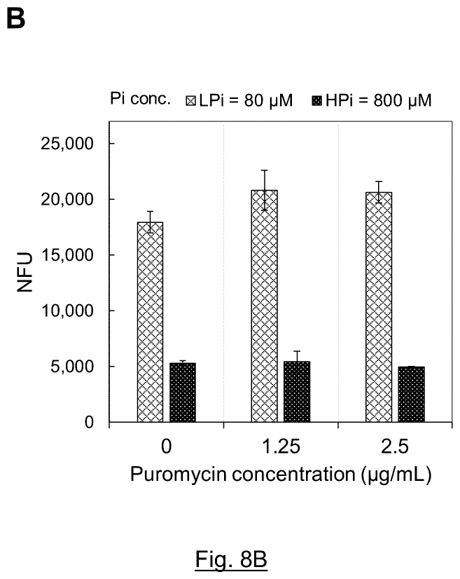

Puromycin is required for selection and maintenance of the expression plasmid; however, its detoxification by puromycin transacetylase requires acetyl-CoA, a central metabolite whose limitation might impose metabolic restraints⁴⁰. That said, the addition of up to 2.5 μg ml⁻¹ puromycin had no significant impact on either cell growth or mCherry expression (FIG. 8). Without wishing to be bound by theory, it is thought that the expression plasmid remained stable during the rather short duration of these experiments.

### Role of BRE, 5′UTR and Start Codon in Phosphate-Dependent Regulation of Ppst.

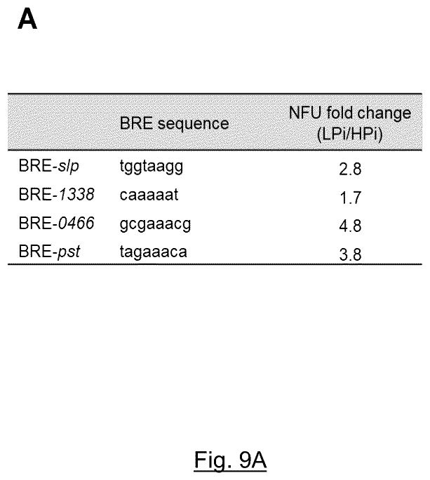

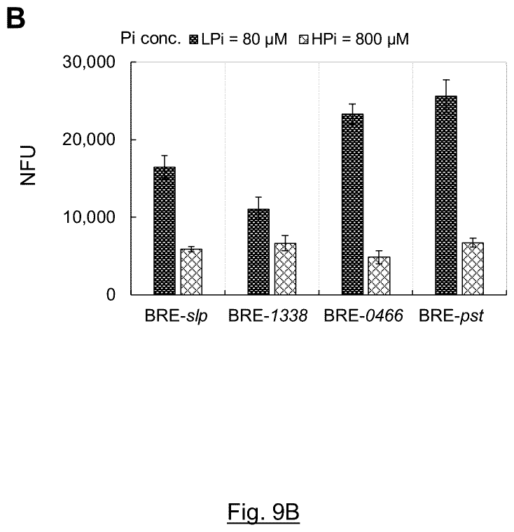

BRE and TATA box sequences are major determinants of transcription efficiency in archaea. Stable binding of the TATA binding protein (TBP) to the highly conserved TATA element requires the concomitant binding of the transcription factor B protein (TFB) to the BRE. Consequently, weak binding of the TBP to the TATA box can be compensated by a strong TFB-BRE interaction or the presence of activators to recruit TBP. On the other hand, a weak TFB-BRE can only be compensated by an activator. There are no reported specific BRE consensus sequences for methanogens. However, in archaea generally, positions −3 and −6 of a 6-7 bp BRE relative to the TATA box have the strongest specificity determinants. To test the effect of BRE on the strength of the pst promoter, the native BRE sequence was replaced by sequences from the promoter of a highly transcribed gene, slp (BRE-slp), and two weakly transcribed genes, MMP1338 and MMP0466 (BRE-1338 and BRE-0466, respectively) (FIG. 9). The native and mutated BREs all retained A at the −3 bp upstream of the TATA box, and only BRE-1338 lacked G at −6 bp. Expression from BRE-0466 was similar to that of the native promoter. By contrast, expression from the other promoters was significantly lower during phosphate limitation. These results suggested that the BRE in Ppst was not a strong determinant of mCherry expression. Presumably, either transcription initiation was not limiting protein expression or other proteins compensated for mutations in the BRE.

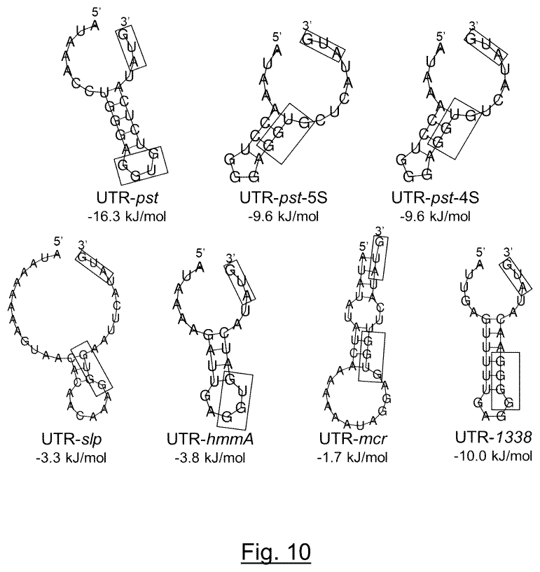

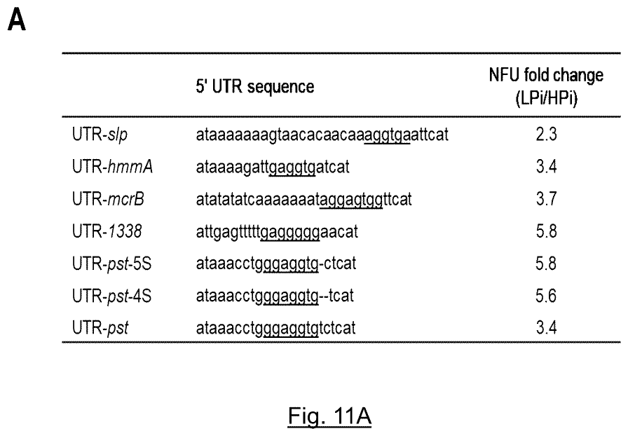

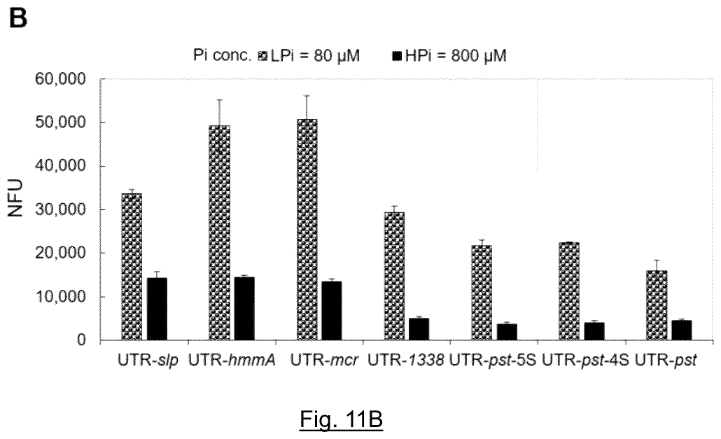

Next, the impact of translation initiation on mCherry expression was studied. First, the effect of 5′ UTR on protein production from Ppst was investigated. The 5′ UTR can influence transcript stability, translation efficiency and protect mRNA from ribonuclease attack. Using the RNAfold program, the secondary structure of the native Ppst 22 bp 5′ UTR (UTR-pst) was predicted. The minimum free energy of folding (ΔG) was −16.3 kJ/mol, and the RBS was within a predicted stem-loop structure (FIG. 10). Spacer lengths between the RBS and start codon of 6-7 bp are optimal for efficient translation, and reduction of the spacer length in the UTR-pst from 6 bp to 4 bp (UTR-pst-4S) and 5 bp (UTR-pst-5S) both weakened and shifted the location of the stem-loop structure (FIG. 10). These mutations significantly increased expression under low Pi conditions by 1.4-fold and increased the expression ratio between limiting and replete conditions from 3.5-fold to ˜6-fold (FIG. 11). Efficient translation requires a tradeoff between mRNA stability and access of the ribosome to the RBS. Highly stable mRNA transcripts degrade slowly, but the ribosome may need more energy to unwind the hairpin for translation initiation. Therefore, increased mCherry expression in UTR-pst-5S and UTR-pst-4S suggested that translation initiation was an important factor in protein expression.

Similarly, the stability of the hairpins in UTR-slp (−3.3 kJ/mol), UTR-hmmA (−3.8 kJ/mol) and UTR-mcr (−1.7 kJ/mol) were all weaker than that in UTR-pst (FIG. 10). For these UTRs from highly expressed genes, the mCherry expression in both limiting and replete phosphate conditions increased by 1.7- to 2.5-fold (FIG. 11). This observation was consistent with a role of the hairpin in the native promoter of lowering expression. Lastly, UTR-1338 possessed a hairpin of intermediate strength between the strong and native promoters. mCherry expression was elevated under the low phosphate conditions but somewhat lower under high phosphate conditions. The result was a nearly 6-fold change in mCherry expression between limiting and replete Pᵢ concentrations, comparable to the UTR-pst-5S and UTR-pst-4S promoters. As a consequence, modifications of the UTR of the Ppst promoter provide means for either increased protein overexpression or increasing the regulation of protein production.

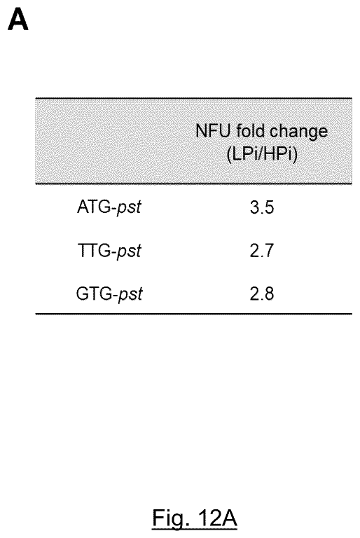

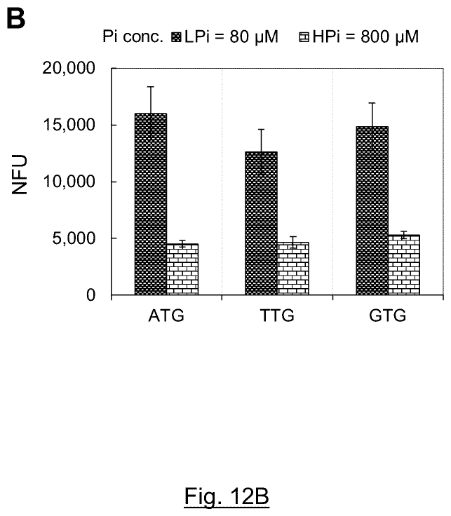

The effect of alternative start codons on translational efficiency of mCherry was also examined. The predominant start codon in most archaeal genomes is ATG, constituting 70-90% of predicted start codons; however, TTG and GTG are also common start codons. In fact, TTG is the start codon of MMP1095, the first ORF of the native pst operon. The ATG start codon of mCherry was replaced with TTG and GTG. During phosphate limitation, there was no significant difference in mCherry NFU between ATG and the alternative start codons TTG and GTG (FIG. 12). For comparison, in Methanosarcina acetivorans the β-glucuronidase activity was similar for start codons ATG and GTG but about 70% lower for TTG. Apparently, the choice of start codon did not greatly impact the overall expression of mCherry from Ppst.

### Example 2: Production of Metabolic Enzymes

To test the Ppst gene expression system, expression vectors were constructed for recombinant mmpX and mcrBDCGA in M. maripaludis.

### Heterologous Expression of MMP10.

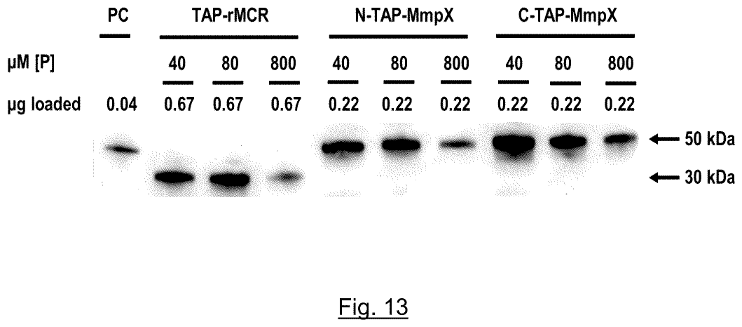

The mmpX gene encodes the methanogen marker protein 10, an S-adenosyl methionine-dependent arginine methylase responsible for the methyl-Arg posttranslational modifications of methylcoenzyme M reductase (which can conveniently be termed Mcr or MCR, and which is referred to in this Example as MCR). Under control of the PhmvA promoter, only low levels of the recombinant protein were expressed in M. maripaludis, presumably because of its toxicity. By contrast, both C-terminal and N-terminal FLAG-tagged MmpXs were expressed at high levels using the 243 bp Ppst following growth at low phosphate concentrations (FIG. 13). Presumably the toxic effects of expressing the recombinant genes such as mmpX were eliminated as expression was turned on between the mid-exponential phase and early stationary phase.

### Heterologous Expression of MCRₐₑₒ.

The gene operon mcrBDCGA encodes methylcoenzyme M reductase (MCR). MCR is the enzyme catalyzing the terminal step in methanogenesis. This complex enzyme contains the nickel tetrapyrrole coenzyme F₄₃₀ as a prosthetic group and possesses multiple unusual posttranslational modifications.

The 243-bp Ppst was introduced into the pMEV5 shuttle vector for heterologous expression of the Methanococcus aeolicus methylcoenzyme M reductase (MCRₐₑₒ) enzyme complex. The recombinant MCR was provided with a tandem affinity purification (TAP) tag comprising of a 3×-FLAG and Twin-Strep tags on the N-terminal of the gamma subunit.

Following growth in formate medium under low and high phosphate concentrations, the relative expression levels of the recombinant MCR were determined by Western blots of cell free extracts of M. maripaludis. Following growth in 40 and 80 μM phosphate, MCR was expressed 2.6 and 3.3-fold higher, respectively, than following growth in 800 μM phosphate (FIG. 13).

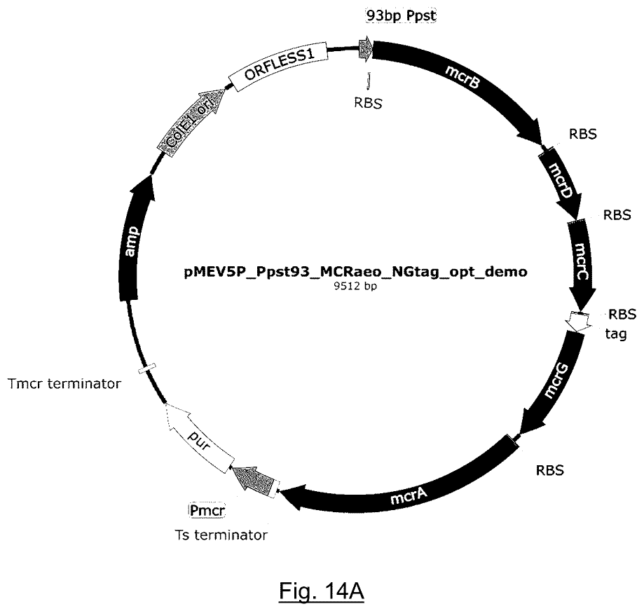

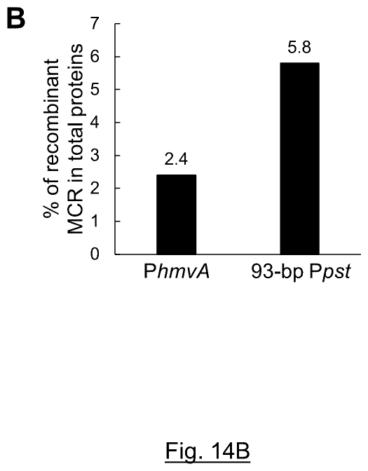

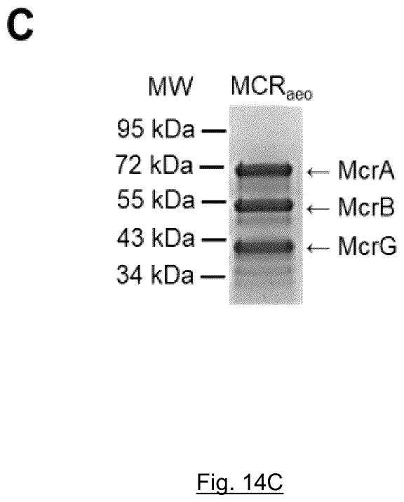

The 93-bp Ppst was introduced into the pMEV5 shuttle vector for heterologous expression of the Methanococcus aeolicus methylcoenzyme M reductase (MCRₐₑₒ) enzyme complex (FIG. 14A). At the highest levels of expression under 80 μM phosphate condition, the recombinant Methanococcus aeolicus MCR (MCRₐₑₒ) expressed in M. maripaludis represented 5.8% of total protein in cell-free extracts, which was a substantial improvement over the expression using the constitutive promoter PhmvA (FIG. 14B). The purified recombinant MCRs contained McrA, B and G subunits, suggesting a fully assembled and functional MCR complex (FIG. 14C). These results demonstrate that Ppst improves expression of recombinant enzymes that are potentially inhibitory to growth. Thus, the Ppst system is useful in production of recombinant metabolic enzymes involved in natural and engineered pathways.

### Examples: Summary

In summary, an inorganic phosphate-regulated gene expression system for heterologous protein production in M. maripaludis was studied using the mCherry reporter assay, and a 3- to 4-fold increase in protein production was achieved upon phosphate limitation. This fold-change was further increased to 6 with comparable overall mCherry expressions when translation initiation was optimized via changes to the 5′ UTR of Ppst. Other (alternative) changes to the 5′-UTR increased overall mCherry expression by 2.5-fold while maintaining the same 3- to 4-fold change at limiting and replete Pᵢ concentrations, suggesting translation initiation plays an important and tunable role in protein expression. The optimal growth conditions for increased gene expression were found to be 80-150 μM initial phosphate concentration. Recombinant MCR expression of ca. 5% of total protein was observed in limiting phosphate concentrations. This expression system, using an inducible promoter, also overcomes the growth burden associated with the toxicity of expressing MmpX with a constitutive promoter, since it largely increases expression late in growth, after biomass has been accumulated.

### SEQUENCES

Set forth below are sequences disclosed herein together with their SEQ ID Nos where applicable.

| Name: | Sequence (DNA or RNA): | SEQ ID No: |
|---|---|---|
| 93 bp | TTTATTCATAAAATAGGAACTAAAATACCAAAATA | SEQ ID No. 1 |
| promoter | GAAACATTTATATACATTTGATGTGTAGGGTATACC | |
| | ATAAACCTGGGAGGTGTCTCAT | |
| 243 bp | GTTGAATCCTCCATTCGTTCAATTCATGTTTCTTCTA | SEQ ID No. 2 |
| promoter | TCAAAAATAACTATGATTACAATTTCTATTTTATAC | |
| | ATCTCAGATATATAATATTTTGTAGGGCTCATTTCG | |
| | GTTCGGTAATTTACTAATTTAATTTTTTAAAATAAC | |
| | TTTAATTTATTCATAAAATAGGAACTAAAATACCAA | |
| | AATAGAAACATTTATATACATTTGATGTGTAGGGTA | |
| | TACCATAAACCTGGGAGGTGTCTCAT | |
| AT rich | TTTATTCATAAAATAGGAACTAAAA | SEQ ID No. 3 |
| region | | |
| native | ATAAACCTGGGAGGTGTCTCAT | SEQ ID No. 4 |
| UTR-pst | | |
| UTR-pst-5S | ATAAACCTGGGAGGTGCTCAT | SEQ ID No. 5 |
| UTR-pst-4S | ATAAACCTGGGAGGTGTCAT | SEQ ID No. 6 |

Table 2 sequences

| | | |
|---|---|---|
| 0mT-F | ATAAAAAACGCCCTATTCGTAATATACTATTC | SEQ ID No. 7 |
| 0mT-R | CTGCAGCGGCCGCTACTAGTT | SEQ ID No. 8 |
| 5p-F346 | ATGGTTTCAAAAGGAGAAGAAGATA | SEQ ID No. 9 |
| 5p-R46 | CTAGAACCGGGCGCGAATTC | SEQ ID No. 10 |
| 5i-F4 | GGGAAAAAGCTTGAATCCTCCATTCGTTCAATTC | SEQ ID No. 11 |
| 5i-R346 | ATGAGACACCTCCCAGGTTTAT | SEQ ID No. 12 |
| P1-F | GGGAAATCTAGATTTATTCATAAAATAGGAACTAA | SEQ ID No. 13 |
| | AATACCAAAATAGAAAC | |
| P2-F | GGGAAATCTAGACAAAATAGAAACATTTATATACA | SEQ ID No. 14 |
| | TTTGATGTGTAGGG | |
| P1-R | GGGAAAGTCTAGAACCGGGCGCGAATT | SEQ ID No. 15 |
| SL1-F | CTCATATGGTTTCAAAAGGAG | SEQ ID No. 16 |
| SL2-F | TCATATGGTTTCAAAAGGAGAAG | SEQ ID No. 17 |
| SL1-R | CACCTCCCAGGTTTATGG | SEQ ID No. 18 |
| SC1-F | GGTGTCTCATTTGGTTTCAAAAG | SEQ ID No. 19 |
| SC2-F | GGTGTCTCATGTGGTTTCAAAAG | SEQ ID No. 20 |
| SC1-R | TCCCAGGTTTATGGTATAC | SEQ ID No. 21 |
| Vector-F | ACTAGTAGCGGCCGCTGCAGGATATA | SEQ ID No. 22 |
| Vector-R | ATGAGACACCTCCCAGGTTTATGGTATACC | SEQ ID No. 23 |
| BDC-F | AAACCTGGGAGGTGTCTCATATGGTAAAGTATGAA | SEQ ID No. 24 |
| | GATAAGATAAG | |
| BDC-R | TAAGATCACCTTAACCTAAATGTTTTTTTATGCTTTC | SEQ ID No. 25 |
| GA-F | AAAGTCAGGTATGGCATACACGCCTCAG | SEQ ID No. 26 |
| GA-R | CTGCAGCGGCCGCTACTAGTTTATTTAGCAGGTAAG | SEQ ID No. 27 |
| | ATAACGTCTC | |
| TAP-ATG-F | GGTGATCTTATGGACTACAAGGACCACGACGGC | SEQ ID No. 28 |
| TAP-ATG-R | ACCTGACTTTTCGAACTGGGGGTGGCT | SEQ ID No. 29 |
| TAP-F | TTTAGGTTAAGGTGATCTTATGGACTAC | SEQ ID No. 30 |
| TAP-R | TGTATGCCATACCTGACTTTTCGAACTG | SEQ ID No. 31 |
| N-mmpX-F | AAAGTCAGGTATGAACCAAAAGTTTGGATTTAATA | SEQ ID No. 32 |
| | ATATTG | |
| N-mmpX-R | CTGCAGCGGCCGCTACTAGTTTAAATAGGATTTCCA | SEQ ID No. 33 |
| | AAGAAGTTTATTTTTTC | |
| N-TAP-F | AAACCTGGGAGGTGTCTCATATGGACTACAAGGAC | SEQ ID No. 34 |
| | CAC | |
| N-TAP-R | TTTGGTTCATACCTGACTTTTCGAACTG | SEQ ID No. 35 |
| C-mmpX-F | AAACCTGGGAGGTGTCTCATATGAACCAAAAGTTT | SEQ ID No. 36 |
| | GGATTTAATAATATTG | |
| C-mmpX-R | CCTTGTAGTCAATAGGATTTCCAAAGAAGTTTATTT | SEQ ID No. 37 |
| | TTTC | |
| C-TAP-F | AAATCCTATTGACTACAAGGACCACGAC | SEQ ID No. 38 |
| C-TAP-R | CTGCAGCGGCCGCTACTAGTTTACTTTTCGAACTGG | SEQ ID No. 39 |
| | GG | |
| mCherry-F | CCATACGAAGGTACACAGACAGCAA | SEQ ID No. 40 |
| mCherry-R | TCTGCTGGGTGTTTAACGTATGC | SEQ ID No. 41 |

FIG. 2 sequences

| | | |
|---|---|---|
| M. aeolicus_ | ATTTTTTCTCCTCTCTTGATATGGGAACTATATACTA | SEQ ID No. 42 |
| Nankai3 | TGTAGCTCATAGTTTTTAATATTATCTTATAATTATA | |
| | TGTGCACCTATATTATATAGCTCATAGTTTTTAAAT | |
| | ATTTCTTTTATTCATATTATATGAACTAAATAATAC | |
| | AAAATAGAAACATTTATATATTAGTATAACGACAG | |
| | GATTATTTAGGCATATACCTGCAATACACAACAGC | |
| | GTTAATAATAAATATGCCATGTTATGTATGGTAAAT | |
| | CGCATACAATAATACAATAATTATAATTGAGGTGA | |
| | TGAA | |
| M. voltae_A3 | ATTATATAATAATATACATTTTATTTTTCTTTTTTGT | SEQ ID No. 43 |
| | ATTTTTAATTTTATATTTTTAATTTTATCTTTTTATAA | |
| | TTATATAATTTAATTCATAAAATAGGAAATATAATT | |
| | CATATATAGAAACATTTATATACAATATTGTACACT | |
| | AAAGTTCTTTAGACATATATTAAAATTCCTAAAATT | |
| | CCTATCAATAAAATATTAAAAATAAGACCACAAAA | |
| | ATCAATAAATCAATAGGTGATTTAG | |
| M. vannielii_ | TTTTAAATCCTCCATGGGCGCTTTTCACGTTTCTTTA | SEQ ID No. 44 |
| SB | ATAAAAATAACTAAAATCACATTTTCTATTTACACT | |
| | ACTCAGATATATAATATTTTGTAGAGTCAATTTACC | |
| | TTTTCGATAATATTTTCCATTTTAAATTATGGTTAGA | |
| | TATGGGTAGTTAAAAAACAGCTAAAAACAATTTAA | |
| | GGAGAAATTAAAAGAAATTAAAAGAAATGAAATA | |
| | TAGTTTAAGAAAATTTAAGAGATAATTATCTATAAA | |
| | ATAGCAGTTAAAACTAAAATTCTAAAAATTTTATTT | |
| | TTATTTTATTTTATTTTATTTTTACTTTTTGTATTATA | |
| | TAATTAATAAAAAAATATTTTATTCATATAATATGA | |
| | ACTAAAGTACCATTTTAGAAACATTTATATACATTT | |
| | AACGTATAGGGTATAACATGGAAAATATTTGAGGT | |
| | GTTACTT | |
| M. maripaludis_ | TGTTGAATCCTCCATTCGTTCAATTCATGTTTCTTCT | SEQ ID No. 45 |
| S2 | ATCAAAAATAACTATGATTACAATTTCTATTTTATA | |
| | CATCTCAGATATATAATATTTTGTAGGGCTCATTTC | |
| | GGTTCGGTAATTTACTAATTTAATTTTTTAAAATAA | |
| | CTTTAATTTATTCATAAAATAGGAACTAAAATACCA | |
| | AAATAGAAACATTTATATACATTTGATGTGTAGGGT | |
| | ATACCATAAACCTGGGAGGTGTCTCCTT | |
| M. maripaludis_ | TGTTGAATCCTCCATTCGTTCAATTCATGTTTCTTCT | SEQ ID No. 46 |
| X1 | ATCAAAAATAACTATGATTACAATTTCTATTTTATA | |
| | CATCTCAGATATATAATATTTTGTAGGGCTCATTTC | |
| | GGTTCGGTAATTTACTAATTTAATTTTTTAAAATAC | |
| | ACTTAATTTATTCATAAAATAGGAACTAAAATACC | |
| | AAAATAGAAACATTTATATACATTTGATGTGTAGG | |
| | GTATATCATAAACCTGGGAGGTGTCTCCTT | |
| M. maripaludis_ | TGTTGAATCCTCCATTCGTTCAAGTCATGTTTCTTCT | SEQ ID No. 47 |
| C6 | ATCAAAAATACCTATGATTACAATTTTATTTTATAC | |
| | GTCTCAGATATATAATATTTTGTAGAGTTAATTTCG | |
| | GTCTTATAATTTAATAAATAAATTATTTTTTTAAAA | |
| | TATACCTATTTATTCATAAAGTAGGAACTAAAATAC | |
| | CAAAATAGAAACATTTATATACAGTTGATGTATAG | |
| | GGTATATCATAAACCTAGGAGGTGTCTCCTT | |
| M. maripaludis_ | TGTTGAATCCTCCATTCATTCGAGTCATGTTTCTTCT | SEQ ID No. 48 |
| C5 | ATCAAAAATAACTATGATTACAATTCTATTTTATAC | |
| | ATCTCAGATATATAATATTTTGTAGGGTTGATTTCA | |
| | GACTTAGTTTTTGATAATTTATTTTTTTAAAATATAT | |
| | TTGAGTTATTCATAAAATAGGAACTAAAATACCAA | |
| | AATAGAAACATTTATATACATTTGATGTGTAGGGC | |
| | ATATCATAAACCTAGGAGGTGCCTCCTT | |
| M. maripaludis_ | TGTTGAATCCTCCATTCGTTCGAGTCATGTTTCTTCT | SEQ ID No. 49 |
| C7 | ATCAAAAATAACTATGATTACAATTCTATTTTATAC | |
| | ATCTCAGATATATAATATTTTGTAGAGTTGATTTCA | |
| | GACTTAGTTTTTGATAATTTATTTTTTTTAAATTAAC | |
| | CTAGTTTATTCATAAAATAGGAACTAAAATACCAA | |
| | AATAGAAACATTTATATACATTTGACGTGTACGGTA | |
| | TATCATAAACCTGGGAGGTGTCTCCTT | |

FIG. 5 sequences

| | | |
|---|---|---|
| 67 bp Ppst | ACCAAAATAGAAACATTTATATACATTTGATGTGTA | SEQ ID No. 50 |
| | GGGTATACCATAAACCTGGGAGGTGTCTCAT | |
| 73 bp Ppst | TAAAATACCAAAATAGAAACATTTATATACATTTG | SEQ ID No. 51 |
| | ATGTGTAGGGTATACCATAAACCTGGGAGGTGTCT | |
| | CAT | |
| 80 bp Ppst | TAGGAACTAAAATACCAAAATAGAAACATTTATAT | SEQ ID No. 52 |
| | ACATTTGATGTGTAGGGTATACCATAAACCTGGGA | |
| | GGTGTCTCAT | |
| 85 bp Ppst | TAAAATAGGAACTAAAATACCAAAATAGAAACATT | SEQ ID No. 53 |
| | TATATACATTTGATGTGTAGGGTATACCATAAACCT | |
| | GGGAGGTGTCTCAT | |

FIG. 9A sequences

| | |
|---|---|
| BRE-slp | TGGTAAGG |
| BRE-1338 | CAAAAAT |
| BRE-0466 | GCGAAACG |
| BRE-pst | TAGAAACA |

FIG. 10 sequences

| | | |
|---|---|---|
| UTR-pst | AUAAACCUGGGAGGUGUCUCAUAUG | SEQ ID No. 54 |
| (RNA) | | |
| UTR-pst-5S | AUAAACCUGGGAGGUGCUCAUAUG | SEQ ID No. 55 |
| (RNA) | | |
| UTR-pst-4S | AUAAACCUGGGAGGUGUCAUAUG | SEQ ID No. 56 |
| (RNA) | | |
| UTR-slp | AUAAAAAAAGUAACACAACAAAGGUGAAUUCAUA | SEQ ID No. 57 |
| (RNA) | UG | |
| UTR-hmmA | AUAAAAGAUUGAGGUGAUCAUAUG | SEQ ID No. 58 |
| (RNA) | | |
| UTR-mcr | AUAUAUAUCAAAAAAAUAGGAGUGGUUCAUAUG | SEQ ID No. 59 |
| (RNA) | | |
| UTR-1338 | AUUGAGUUUUUGAGGGGGAACAUAUG | SEQ ID No. 60 |
| (RNA) | | |

FIG. 11A sequences

| | | |
|---|---|---|
| UTR-slp | ATAAAAAAAGTAACACAACAAAGGTGAATTCAT | SEQ ID No. 61 |
| UTR-hmmA | ATAAAAGATTGAGGTGATCAT | SEQ ID No. 62 |
| UTR-mcr | ATATATATCAAAAAAATAGGAGTGGTTCAT | SEQ ID No. 63 |
| UTR-1338 | ATTGAGTTTTTGAGGGGGAACAT | SEQ ID No. 64 |
| UTR-pst-5S | ATAAACCTGGGAGGTGCTCAT | SEQ ID No. 4 |
| UTR-pst-4S | ATAAACCTGGGAGGTGTCAT | SEQ ID No. 5 |
| UTR-pst | ATAAACCTGGGAGGTGTCTCAT | SEQ ID No. 6 |

FIG. 12A sequences

| | | |
|---|---|---|
| ATG-pst | TTTATTCATAAAATAGGAACTAAAATACCAAAATA | SEQ ID No. 65 |
| | GAAACATTTATATACATTTGATGTGTAGGGTATACC | |
| | ATAAACCTGGGAGGTGTCTCATATG | |
| TTG-pst | TTTATTCATAAAATAGGAACTAAAATACCAAAATA | SEQ ID No. 66 |
| | GAAACATTTATATACATTTGATGTGTAGGGTATACC | |
| | ATAAACCTGGGAGGTGTCTCATTTG | |
| GTG-pst | TTTATTCATAAAATAGGAACTAAAATACCAAAATA | SEQ ID No. 67 |
| | GAAACATTTATATACATTTGATGTGTAGGGTATACC | |
| | ATAAACCTGGGAGGTGTCTCATGTG | |

Many alterations, modifications, and variations will be apparent to those skilled in the art in light of the foregoing description without departing from the spirit or scope of the present disclosure.

When numerical lower limits and numerical upper limits are listed herein, ranges from any lower limit to any upper limit are contemplated.

The invention claimed is:

1. An isolated DNA molecule, comprising a sequence having at least about 80% identity to at least about 80 contiguous nucleotides of SEQ ID No. 1.

2. An isolated DNA molecule comprising a sequence having at least about 80% identity to at least about 200 contiguous nucleotides of SEQ ID No. 2.

3. An expression cassette comprising:
a promoter comprising the DNA molecule according to claim 1 or claim 2; and
a gene encoding a polypeptide or a functional RNA sequence, wherein the gene is operably linked to the promoter.

4. The expression cassette according to claim 3, wherein the gene encodes an enzyme associated with methanogenesis or reverse methanogenesis.

5. A vector comprising an expression cassette according to claim 3.

6. The vector according to claim 5, further comprising one or more selectable markers and/or reporter genes.

7. A prokaryotic host cell comprising the expression cassette according to claim 3.

8. The prokaryotic host cell according to claim 7, wherein the cell is an archaeal cell.

9. The prokaryotic host cell according to claim 8, wherein the cell is a methanogenic archaeal cell.

10. The prokaryotic host cell according to claim 9, wherein the cell is a Methanococcus cell.

11. The prokaryotic host cell according to claim 9, wherein the cell is a cell of: Methanobacterium bryantii; Methanobacterium formicum; Methanobrevibacter arboriphilicus; Methanobrevibacter gottschalkii; Methanobrevibacter ruminantium; Methanobrevibacter smithii; Methanocalculus chunghsingensis; Methanococcoides burtonii; Methanococcus aeolicus; Methanococcus voltae; Methanocaldococcus jannaschii; Methanococcus maripaludis; Methanococcus vannielii; Methanocorpusculum labreanum; Methanoculleus bourgensis (Methanogenium olentangyi & Methanogenium bourgense); Methanoculleus marisnigri; Methanoflorens stordalenmirensis; Methanofollis liminatans; Methanogenium cariaci; Methanogenium frigidum; Methanogenium organophilum; Methanogenium wolfei; Methanomicrobium mobile; Methanopyrus kandleri; Methanoregula boonei; Methanosaeta concilii; Methanosaeta thermophila; Methanosarcina acetivorans; Methanosarcina barkeri; Methanosarcina mazei; Methanosphaera stadtmanae; Methanospirillium hungatei; Methanothermobacter defluvii; Methanothermobacter thermautotrophicus; Methanothermobacter marburgensis; Methanothermobacter thermoflexus; Methanothermobacter wolfei; or Methanothrix soehngenii.

12. The prokaryotic host cell according to claim 10, wherein the cell is a Methanococcus maripaludis cell.

13. A cell culture comprising prokaryotic host cells according to claim 7.

14. A method for inducing expression of an exogenous gene in a prokaryotic cell culture according to claim 13, the method comprising lowering the concentration of inorganic phosphates in the culture from an initial concentration.

15. The method according to claim 14, wherein the concentration of inorganic phosphate is lowered from an initial concentration in a range of from about 10 to 300 μM.

16. The method according to claim 15, wherein the concentration of inorganic phosphate is lowered from an initial concentration in a range of from about 80 to 150 μM.

17. A method for transforming a prokaryotic cell, the method comprising:
introducing a vector according to claim 5 into the prokaryotic cell; and
selecting for a transformed prokaryotic cell.

18. A method for co-transforming a prokaryotic cell, the method comprising:
introducing an expression cassette according to claim 3 and a nucleic acid sequence encoding a selectable marker into the prokaryotic cell; and
selecting for the presence of the selectable marker in a transformed prokaryotic cell to provide a prokaryotic cell transformed with the expression cassette.


SEQUENCE LISTING (67 sequences)

SEQ ID NO: 1 - DNA, 93 bp, Methanococcus maripaludis
tttattcataaaataggaactaaaataccaaaatagaaacatttatatacatttgatgtgtagggtataccataaacctgggaggtgtctcat

SEQ ID NO: 2 - DNA, 243 bp, Methanococcus maripaludis
gttgaatcctccattcgttcaattcatgtttcttctatcaaaaataactatgattacaatttctattttatacatctcagatatataatattttgtagggctcatttcggttcggtaatttactaatttaattttttaaaataactttaatttattcataaaataggaactaaaataccaaaatagaaacatttatatacatttgatgtgtagggtataccataaacctgggaggtgtctcat

SEQ ID NO: 3 - DNA, 25 bp, Methanococcus maripaludis
tttattcataaaataggaactaaaa

SEQ ID NO: 4 - DNA, 22 bp, Methanococcus maripaludis
ataaacctgggaggtgtctcat

SEQ ID NO: 5 - DNA, 21 bp, Artificial sequence
  UTR-pst-5S
ataaacctgggaggtgctcat

SEQ ID NO: 6 - DNA, 20 bp, Artificial sequence
  UTR-pst-4S
ataaacctgggaggtgtcat

SEQ ID NO: 7 - DNA, 32 bp, Artificial sequence
  Primer - 0mT-F
ataaaaaacgccctattcgtaatatactattc

SEQ ID NO: 8 - DNA, 21 bp, Artificial sequence
  Primer - 0mT-R
ctgcagcggccgctactagtt

SEQ ID NO: 9 - DNA, 25 bp, Artificial sequence
  Primer - 5p-F346
atggtttcaaaaggagaagaagata

SEQ ID NO: 10 - DNA, 20 bp, Artificial sequence
  Primer - 5p-R46
ctagaaccgggcgcgaattc

SEQ ID NO: 11 - DNA, 34 bp, Artificial sequence
  Primer - 5i-F4
gggaaaaagcttgaatcctccattcgttcaattc

SEQ ID NO: 12 - DNA, 22 bp, Artificial sequence
  Primer - 5i-R346
atgagacacctcccaggtttat

SEQ ID NO: 13 - DNA, 52 bp, Artificial sequence
  Primer - P1-F
gggaaatctagatttattcataaaataggaactaaaataccaaaatagaaac

SEQ ID NO: 14 - DNA, 49 bp, Artificial sequence
  Primer - P2-F
gggaaatctagacaaaatagaaacatttatatacatttgatgtgtaggg

SEQ ID NO: 15 - DNA, 27 bp, Artificial sequence
  Primer - P1-R
gggaaagtctagaaccgggcgcgaatt

SEQ ID NO: 16 - DNA, 21 bp, Artificial sequence
  Primer - SL1-F
ctcatatggtttcaaaaggag

SEQ ID NO: 17 - DNA, 23 bp, Artificial sequence
  Primer - SL2-F
tcatatggtttcaaaaggagaag

SEQ ID NO: 18 - DNA, 18 bp, Artificial sequence
  Primer - SL1-R
cacctcccaggtttatgg

SEQ ID NO: 19 - DNA, 23 bp, Artificial sequence
  Primer - SC1-F
ggtgtctcatttggtttcaaaag

SEQ ID NO: 20 - DNA, 23 bp, Artificial sequence
  Primer - SC2-F
ggtgtctcatgtggtttcaaaag

SEQ ID NO: 21 - DNA, 19 bp, Artificial sequence
  Primer - SC1-R
tcccaggtttatggtatac

SEQ ID NO: 22 - DNA, 26 bp, Artificial sequence
  Priimer - Vector-F
actagtagcggccgctgcaggatata

SEQ ID NO: 23 - DNA, 30 bp, Artificial sequence
  Primer - Vector-R
atgagacacctcccaggtttatggtatacc

SEQ ID NO: 24 - DNA, 46 bp, Artificial sequence
  Primer - BDC-F
aaacctgggaggtgtctcatatggtaaagtatgaagataagataag

SEQ ID NO: 25 - DNA, 37 bp, Artificial sequence
  Primer - BDC-R
taagatcaccttaacctaaatgtttttttatgctttc

SEQ ID NO: 26 - DNA, 28 bp, Artificial sequence
  Primer - GA-F
aaagtcaggtatggcatacacgcctcag

SEQ ID NO: 27 - DNA, 46 bp, Artificial sequence
  Primer - GA-R
ctgcagcggccgctactagtttatttagcaggtaagataacgtctc

SEQ ID NO: 28 - DNA, 33 bp, Artificial sequence
  Primer - TAP-ATG-F
ggtgatcttatggactacaaggaccacgacggc

SEQ ID NO: 29 - DNA, 27 bp, Artificial sequence
  Primer - TAP-ATG-R
acctgacttttcgaactgggggtggct

SEQ ID NO: 30 - DNA, 28 bp, Artificial sequence
  Primer - TAP-F
tttaggttaaggtgatcttatggactac

SEQ ID NO: 31 - DNA, 28 bp, Artificial sequence
  Primer - TAP-R
tgtatgccatacctgacttttcgaactg

SEQ ID NO: 32 - DNA, 41 bp, Artificial sequence
  Primer - N-mmpX-F
aaagtcaggtatgaaccaaaagtttggatttaataatattg

SEQ ID NO: 33 - DNA, 53 bp, Artificial sequence
  Primer - N-mmpX-R
ctgcagcggccgctactagtttaaataggatttccaaagaagtttattttttc

SEQ ID NO: 34 - DNA, 38 bp, Artificial sequence
  Primer - N-TAP-F
aaacctgggaggtgtctcatatggactacaaggaccac

SEQ ID NO: 35 - DNA, 28 bp, Artificial sequence
  Primer - N-TAP-R
tttggttcatacctgacttttcgaactg

SEQ ID NO: 36 - DNA, 51 bp, Artificial sequence
  Primer - C-mmpX-F
aaacctgggaggtgtctcatatgaaccaaaagtttggatttaataatattg

SEQ ID NO: 37 - DNA, 40 bp, Artificial sequence
  Primer - C-mmpX-R
ccttgtagtcaataggatttccaaagaagtttattttttc

SEQ ID NO: 38 - DNA, 28 bp, Artificial sequence
  Primer - C-TAP-F
aaatcctattgactacaaggaccacgac

SEQ ID NO: 39 - DNA, 38 bp, Artificial sequence
  Primer - C-TAP-R
ctgcagcggccgctactagtttacttttcgaactgggg

SEQ ID NO: 40 - DNA, 25 bp, Artificial sequence
  Primer - mCherry-F
ccatacgaaggtacacagacagcaa

SEQ ID NO: 41 - DNA, 23 bp, Artificial sequence
  Primer - mCherry-R
tctgctgggtgtttaacgtatgc

SEQ ID NO: 42 - DNA, 291 bp, Methanococcus aeolicus strain Nankai3
attttttctcctctcttgatatgggaactatatactatgtagctcatagtttttaatattatcttataattatatgtgcacctatattatatagctcatagtttttaaatatttcttttattcatattatatgaactaaataatacaaaatagaaacatttatatattagtataacgacaggattatttaggcatatacctgcaatacacaacagcgttaataataaatatgccatgttatgtatggtaaatcgcatacaataatacaataattataattgaggtgatgaa

SEQ ID NO: 43 - DNA, 243 bp, Methanococcus voltae strain A3
attatataataatatacattttatttttcttttttgtatttttaattttatatttttaattttatctttttataattatataatttaattcataaaataggaaatataattcatatatagaaacatttatatacaatattgtacactaaagttctttagacatatattaaaattcctaaaattcctatcaataaaatattaaaaataagaccacaaaaatcaataaatcaataggtgatttag

SEQ ID NO: 44 - DNA, 439 bp, Methanococcus vannielii strain SB
ttttaaatcctccatgggcgcttttcacgtttctttaataaaaataactaaaatcacattttctatttacactactcagatatataatattttgtagagtcaatttaccttttcgataatattttccattttaaattatggttagatatgggtagttaaaaaacagctaaaaacaatttaaggagaaattaaaagaaattaaaagaaatgaaatatagtttaagaaaatttaagagataattatctataaaatagcagttaaaactaaaattctaaaaattttatttttattttattttattttatttttactttttgtattatataattaataaaaaaatattttattcatataatatgaactaaagtaccattttagaaacatttatatacatttaacgtatagggtataacatggaaaatatttgaggtgttactt

SEQ ID NO: 45 - DNA, 245 bp, Methanococcus maripaludis strain S2
tgttgaatcctccattcgttcaattcatgtttcttctatcaaaaataactatgattacaatttctattttatacatctcagatatataatattttgtagggctcatttcggttcggtaatttactaatttaattttttaaaataactttaatttattcataaaataggaactaaaataccaaaatagaaacatttatatacatttgatgtgtagggtataccataaacctgggaggtgtctcctt

SEQ ID NO: 46 - DNA, 245 bp, Methanococcus maripaludis strain X1
tgttgaatcctccattcgttcaattcatgtttcttctatcaaaaataactatgattacaatttctattttatacatctcagatatataatattttgtagggctcatttcggttcggtaatttactaatttaattttttaaaatacacttaatttattcataaaataggaactaaaataccaaaatagaaacatttatatacatttgatgtgtagggtatatcataaacctgggaggtgtctcctt

SEQ ID NO: 47 - DNA, 247 bp, Methanococcus maripaludis strain C6
tgttgaatcctccattcgttcaagtcatgtttcttctatcaaaaatacctatgattacaattttattttatacgtctcagatatataatattttgtagagttaatttcggtcttataatttaataaataaattatttttttaaaatatacctatttattcataaagtaggaactaaaataccaaaatagaaacatttatatacagttgatgtatagggtatatcataaacctaggaggtgtctcctt

SEQ ID NO: 48 - DNA, 244 bp, Methanococcus maripaludis strain C5
tgttgaatcctccattcattcgagtcatgtttcttctatcaaaaataactatgattacaattctattttatacatctcagatatataatattttgtagggttgatttcagacttagtttttgataatttatttttttaaaatatatttgagttattcataaaataggaactaaaataccaaaatagaaacatttatatacatttgatgtgtagggcatatcataaacctaggaggtgcctcctt

SEQ ID NO: 49 - DNA, 244 bp, Methanococcus maripaludis strain C7
tgttgaatcctccattcgttcgagtcatgtttcttctatcaaaaataactatgattacaattctattttatacatctcagatatataatattttgtagagttgatttcagacttagtttttgataatttattttttttaaattaacctagtttattcataaaataggaactaaaataccaaaatagaaacatttatatacatttgacgtgtacggtatatcataaacctgggaggtgtctcctt

SEQ ID NO: 50 - DNA, 67 bp, Methanococcus maripaludis
accaaaatagaaacatttatatacatttgatgtgtagggtataccataaacctgggaggtgtctcat

SEQ ID NO: 51 - DNA, 73 bp, Methanococcus maripaludis
taaaataccaaaatagaaacatttatatacatttgatgtgtagggtataccataaacctgggaggtgtctcat

SEQ ID NO: 52 - DNA, 80 bp, Methanococcus maripaludis
taggaactaaaataccaaaatagaaacatttatatacatttgatgtgtagggtataccataaacctgggaggtgtctcat

SEQ ID NO: 53 - DNA, 85 bp, Methanococcus maripaludis
taaaataggaactaaaataccaaaatagaaacatttatatacatttgatgtgtagggtataccataaacctgggaggtgtctcat

SEQ ID NO: 54 - RNA, 25 nt, Methanococcus maripaludis
auaaaccugggaggugucucauaug

SEQ ID NO: 55 - RNA, 24 nt, Artificial sequence
  UTR-pst-5S (RNA)
auaaaccugggaggugcucauaug

SEQ ID NO: 56 - RNA, 23 nt, Artificial sequence
  UTR-pst-4S (RNA)
auaaaccugggaggugucauaug

SEQ ID NO: 57 - RNA, 36 nt, Artificial sequence
  UTR-slp (RNA)
auaaaaaaaguaacacaacaaaggugaauucauaug

SEQ ID NO: 58 - RNA, 24 nt, Artificial sequence
  UTR-hmmA (RNA)
auaaaagauugaggugaucauaug

SEQ ID NO: 59 - RNA, 33 nt, Artificial sequence
  UTR-mcr (RNA)
auauauaucaaaaaaauaggagugguucauaug

SEQ ID NO: 60 - RNA, 26 nt, Artificial sequence
  UTR-1338 (RNA)
auugaguuuuugagggggaacauaug

SEQ ID NO: 61 - DNA, 33 bp, Artificial sequence
  UTR-slp
ataaaaaaagtaacacaacaaaggtgaattcat

SEQ ID NO: 62 - DNA, 21 bp, Artificial sequence
  UTR-hmmA
ataaaagattgaggtgatcat

SEQ ID NO: 63 - DNA, 30 bp, Artificial sequence
  UTR-mcr
atatatatcaaaaaaataggagtggttcat

SEQ ID NO: 64 - DNA, 23 bp, Artificial sequence
  UTR-1338
attgagtttttgagggggaacat

SEQ ID NO: 65 - DNA, 96 bp, Artificial sequence
  ATG-pst
tttattcataaaataggaactaaaataccaaaatagaaacatttatatacatttgatgtgtagggtataccataaacctgggaggtgtctcatatg

SEQ ID NO: 66 - DNA, 96 bp, Artificial sequence
  TTG-pst
tttattcataaaataggaactaaaataccaaaatagaaacatttatatacatttgatgtgtagggtataccataaacctgggaggtgtctcatttg

SEQ ID NO: 67 - DNA, 96 bp, Artificial sequence
  GTG-pst
tttattcataaaataggaactaaaataccaaaatagaaacatttatatacatttgatgtgtagggtataccataaacctgggaggtgtctcatgtg